SEQUENCE LISTING (4 sequences)

SEQ ID NO: 1 - protein, 324 aa, Haemophilus influenzae
MTNIQTQLDNLRKTLRQYEYEYHVLDNPSVPDSEYDRLFHQLKALELEHPEFLTSDSPTQRVGAKPLSGFSQIRHEIPMLSLDNAFSDAEFNAFVKRIEDRLILLPKPLTFCCEPKLDGLAVSILYVNGELTQAATRGDGTTGEDITANIRTIRNVPLQLLTDNPPARLEVRGEVFMPHAGFERLNKYALEHNEKTFANPRNAAAGSLRQLDPNITSKRPLVLNAYGIGIAEGVDLPTTHYARLQWLKSIGIPVNPEIRLCNGADEVLGFYRDIQNKRSSLGYDIDGTVLKINDIALQNELGFISKAPRWAIAYKFPAQEELTL

SEQ ID NO: 2 - DNA, 975 bp, Haemophilus influenzae
atgacaaatattcaaactcaactagacaatctacgcaaaaccttgcgccaatatgaatacgaataccacgttttagataatccgagtgtgcctgatagcgaatacgatcgtttatttcatcagctcaaagccctagaattagagcatcctgaatttctgacgtcagattcgcccactcaacgtgttggtgcaaaaccactttctgggtttagccaaattcgtcacgaaattcctatgctctctttggataatgctttttccgatgcagaatttaatgcttttgtaaaacgcattgaagatcgtttaatcctattaccgaaaccacttactttctgttgcgaacctaaacttgatggcttggctgtgagtattttgtatgttaatggtgaacttacacaagccgccactcgtggtgatggcaccacaggcgaagatattacagccaatatccgcacgattcgtaatgttccattgcaacttttaacagataatcctccagcacgtttagaggtgcggggcgaagtttttatgccgcacgcaggctttgagcgtttaaataaatatgcgttagaacataatgaaaaaacctttgctaatcctcgcaatgcagcggcaggctctttacgccagcttgatcctaatattaccagcaaacgtccgctggtattaaatgcttatggtattggaattgctgagggggttgatctgccgactacgcattatgctcgtttgcaatggctaaaatctatcgggattccagtaaatcctgaaattcgtttatgcaatggtgcagatgaagttttaggtttttatcgagatattcaaaacaaacgtagctcgttaggttatgatattgacggaacggtattaaaaatcaatgatatagccttacaaaatgaactaggatttatttctaaagcacctcgctgggcgattgcttataaattccccgcccaagaagaattaaccctgtag

SEQ ID NO: 3 - DNA, 33 bp, Artificial
  Primer1
ccgagaatcatatgacaaatattcaaactcaac

SEQ ID NO: 4 - DNA, 33 bp, Artificial
  Primer2
aacagaattctacagggttaattcttcttgggc

(19) United States
(12) Patent Application Publication
(10) Pub. No.: US 2008/0262811 A1
(43) Pub. Date: Oct. 23, 2008

(54) Crystal Structure of Haemophilus Influenzae Nad Dependent Dna Ligase and Uses Thereof

(75) Inventors: Sushmita Lahiri, Medway, MA (US); Scott Mills, Framingham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/571,965

(22) PCT Filed: Aug. 9, 2005
(86) PCT No.: PCT/GB2005/003125

§ 371 Date: Jan. 11, 2007

Related U.S. Application Data
(60) Provisional application No. 60/600,667, filed on Aug. 10, 2004.

(51) Int. Cl. G06G 7/48; C12N 11/02

(52) U.S. Cl. 703/11; 435/177

(57) ABSTRACT

The present invention relates crystals of LigA and computer-assisted methods for screening, identifying, and designing inhibitors and allosteric modulators of LigA.

### FIELD OF THE INVENTION

The present invention relates to crystals of DNA ligase A (LigA) from gram negative bacteria and computer-assisted methods for screening, identifying, and designing inhibitors and modulators of LigA.

### BACKGROUND

DNA ligases catalyze the formation of a phosphodiester linkage at single-strand breaks between adjacent 3′-OH and 5′-phosphate termini in double-stranded DNA (Lehman 1974. Science 186: 790-797). This activity plays an indispensable role in DNA replication where it joins Okazaki fragments. DNA ligase also plays a role in repair of damaged DNA and in recombination (Wilkinson 2001. Molecular Microbiology 40: 1241-1248). An early report describing conditional lethal mutations in the DNA ligase gene (ligA) of Escherichia coli supported the essentiality of this enzyme (Dermody et al. 1979. Journal of Bacteriology 139: 701-704). This was followed by the isolation and characterization of DNA ligase temperature-sensitive or knockout mutants of Salmonella typhimurium, Bacillus subtilis, Staphylococcus aureus (Park et al. 1989. Journal of Bacteriology 171: 2173-2180, Kaczmarek et al. 2001. Journal of Bacteriology 183: 3016-3024, Petit and Ehrlich. 2000. Nucleic Acids Research 28: 4642-4648). In all species, DNA ligase was shown to be essential.

The DNA ligase family can be roughly divided into two classes: those requiring ATP for adenylation (eukaryotic cells, viruses and bacteriophages), and those requiring NAD⁺ (nicotinamide adenine dinucleotide) for adenylation, which include all known bacterial DNA ligases (Wilkinson 2001, supra). Eukaryotic, bacteriophage, and viral DNA ligases show little sequence homology to DNA ligases from prokaryotes, apart from a conserved KXDG motif located within the central cofactor-binding core of the enzyme. Amino acid sequence comparisons clearly show that NAD⁺-dependent ligases are phylogenically unrelated to the ATP-dependent DNA ligases. The apparent lack of similarity between bacteria and higher organisms suggests that bacterial DNA ligase may be a good target for selective new antibacterials.

X-ray crystal structures have been reported for the ATP-dependent DNA ligase from T7 phage (Subramanya et al. 1996. Cell 85: 607-615), the N-terminal adenylation domain of B. stearothermophilus DNA ligase (Singleton et al. 1999. Structure 7: 35-42), and the full length Thermus filiformis DNA ligase with AMP covalently bound (Lee et al. 2000. EMBO Journal 19: 1119-1129). Comparison of these structures revealed that, while a core fold and key nucleotide binding residues in the adenylation domains are conserved between both classes of DNA ligases, sequence differences outside of this motif must exist to explain the cofactor specificity.

### SUMMARY

Disclosed herein are the three-dimensional structure of LigA adenylation domain from the H. influenzae bacterium in complex with NAD⁺ and AMP (adenosine monophosphate); binding sites of LigA adenylation domain; methods for identifying and/or designing compounds or agents that bind the LigA adenylation domain, including ligands, drugs, or inhibitors that partially or totally inhibit LigA activity, proteins and small organic molecules that bind LigA; methods for crystallizing LigA adenylation domain; and computer-assisted methods for identifying, screening, and/or designing agents that bind the LigA adenylation domain.

### BRIEF DESCRIPTION OF THE DRAWINGS

FIG. 1 depicts the three-dimensional atomic coordinates of the crystal structure of LigA from H. influenzae complexed with AMP and NAD⁺.

FIG. 2 depicts a ribbon diagram of LigA with ligands AMP and NAD⁺ bound.

### DETAILED DESCRIPTION

The present invention is based upon the crystallization of H. influenzae LigA adenylation domain, and the determination of the crystal structure (three-dimensional structure) of a complex of H. influenzae LigA adenylation domain with AMP and NAD⁺.

Moreover, the present invention is based on the identification of the physiological NAD⁺ binding site on the LigA protein. The NAD⁺ binding site is present near the N-terminal subdomain 1a.

LigA Polypeptides, Crystals and Space Groups

The present invention provides information relating to an isolated polypeptide of a LigA adenylation domain, or a portion of a polypeptide of the LigA adenylation domain, which functions as a binding site when folded in the proper 3-D orientation. As used herein, the term “isolated” in reference to proteins or polypeptides, means a protein, a polypeptide, or a portion thereof, which, by virtue of its origin or manipulation, has been removed from its natural state, or is otherwise not in its natural state. By “isolated” it is further meant a protein or polypeptide that is: (i) synthesized chemically; (ii) expressed in a host cell and purified away from associated and contaminating proteins; or (iii) purified away from associated and contaminating proteins. The term generally means a protein or polypeptide that has been separated from other proteins and nucleic acids with which it naturally occurs. In some embodiments of the present invention, the polypeptide is also separated from substances such as antibodies or gel matrices (for example, polyacrylamide) that are used to purify it.

Each of the isolated polypeptide sequences can be a native sequence of the LigA adenylation domain, or a sequence that is at least 35%, 40%, 45%, 50% 55%, 60%, 65%, 70%, 75%, 80%, 85%, 90%, 95%, 98% or 99% homologous to the amino acid sequence represented by SEQ ID NO:1. In the present invention, “amino acid homology” is a measure of the identity of primary amino acid sequences. In order to characterize the homology, subject sequences are aligned so that the highest percentage homology (match) is obtained, after introducing gaps, if necessary, to achieve maximum percent homology. N- or C-terminal extensions shall not be construed as affecting homology. “Identity” per se has an art-recognized meaning and can be calculated using published techniques. Computer program methods to determine identity between two sequences include, for example, DNAStar® software (DNAStar Inc., Madison, Wis.); the GCG® program package (Devereux et al., 1984, Nucl. Acids Res., 12:387); BLASTP, BLASTN, FASTA (Altschul et al., 1990, J. Mol. Biol., 215:403). Homology (identity or similarity) as defined herein is determined using the computer program, BLAST 2 Sequences (Tatusova and Madden, 1999, FEMS Microbiol. Lett. 174:247-250; available from the NCBI), employing default settings for all parameters, such that percentage identity and/or similarity are calculated over the full length of the aligned sequences, and that gaps in homology of up to about 90% of the total number of nucleotides or amino acids in the reference sequence are allowed.

The isolated LigA adenylation domain can be a variant of the LigA adenylation domain. In one example, the variant may have an amino acid sequence that is different by one or more amino acid substitutions from the sequence disclosed in SEQ ID NO:1. Embodiments which comprise amino acid deletions and/or additions are also contemplated. The variant may have conservative changes (amino acid similarity), wherein a substituted amino acid has structural or chemical properties similar to those of the amino acid residue it replaces (e.g., the replacement of leucine with isoleucine). Guidance in determining which and how many amino acid residues may be substituted, inserted, or deleted without abolishing biological or pharmacological activity may be reasonably inferred in view of this disclosure and may further be found using computer programs well known in the art, for example, DNAStar® software.

Amino acid substitutions may be made, for instance, on the basis of similarity in polarity, charge, solubility, hydrophobicity, hydrophilicity, and/or the amphipathic nature of the residues as long as a biological and/or pharmacological activity of the native molecule is retained.

Example substitutions are set forth in Table 1 as follows:

TABLE 1

| Original Residue | Example conservative substitutions |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

The invention also includes a crystal of the LigA adenylation domain. In one embodiment, the crystal is the LigA adenylation domain complexed with AMP and NAD⁺. The LigA adenylation domain can be from any gram negative or positive bacteria including H. influenzae. In particular, the LigA adenylation domain can be from any bacterium including a gram negative bacterium including Helicobacter pylori, Escherichia coli, and Pseudomonas aeruginosa.

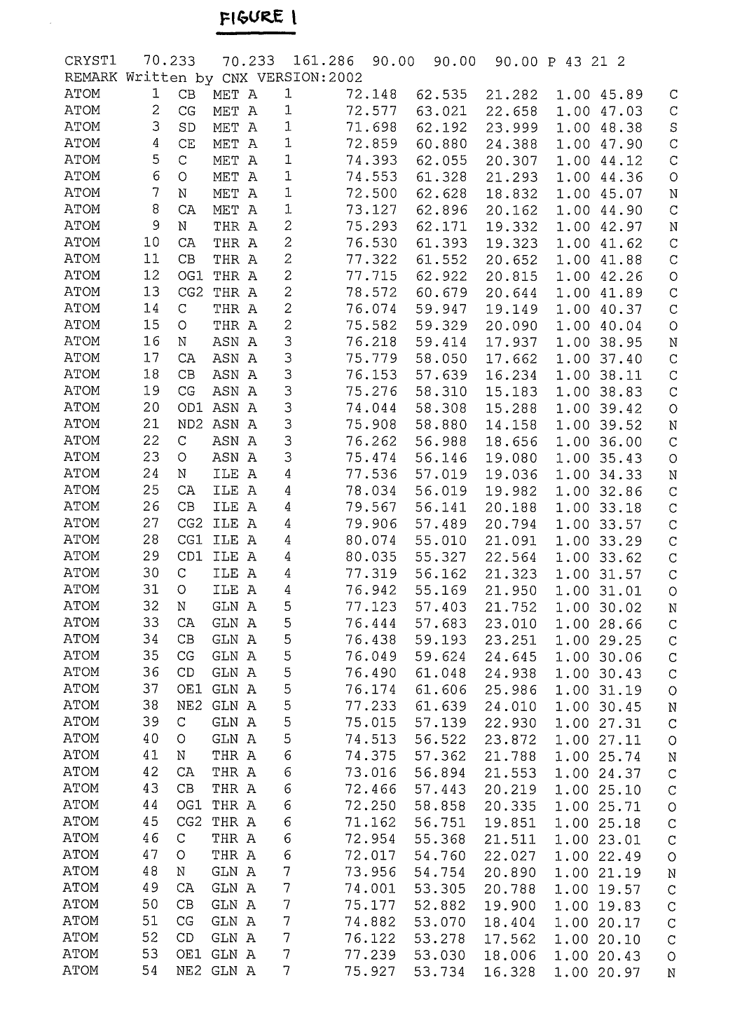

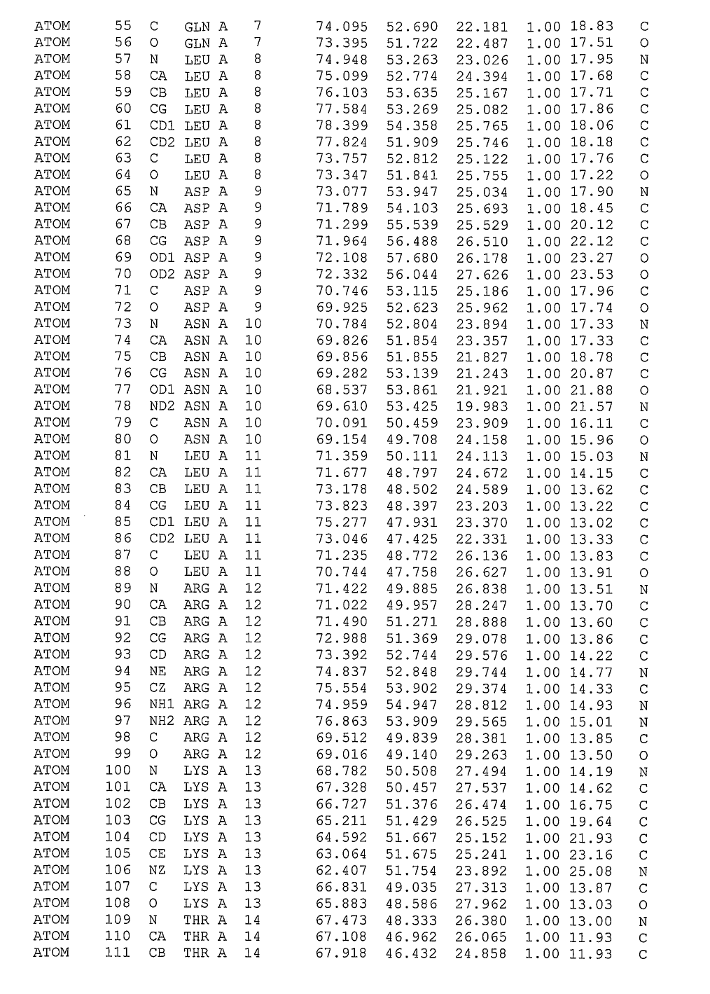

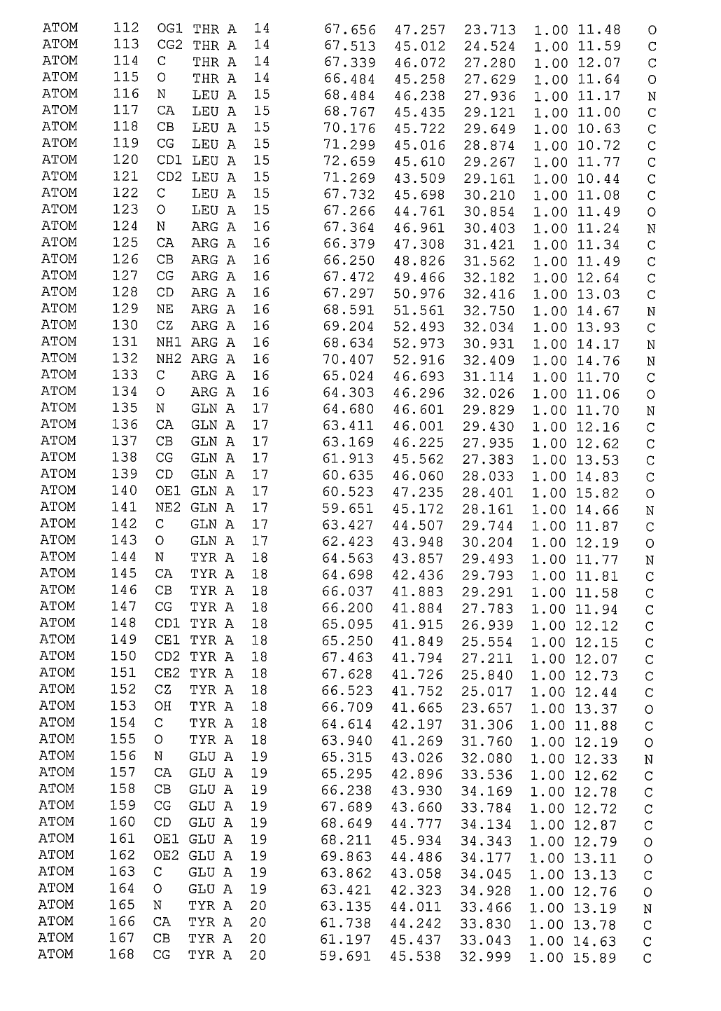

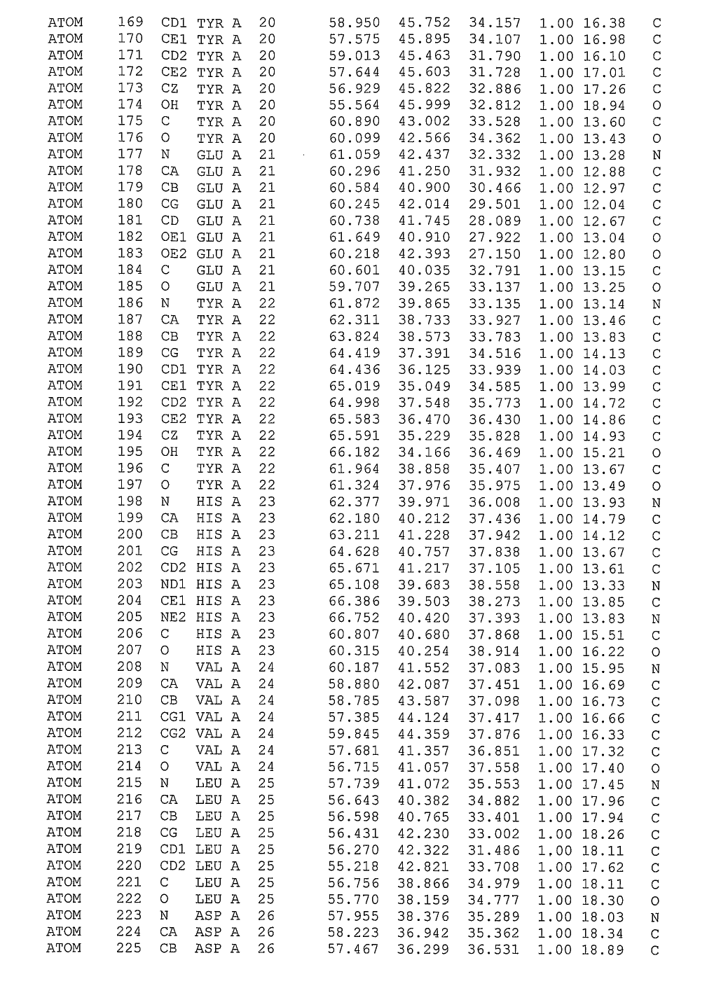

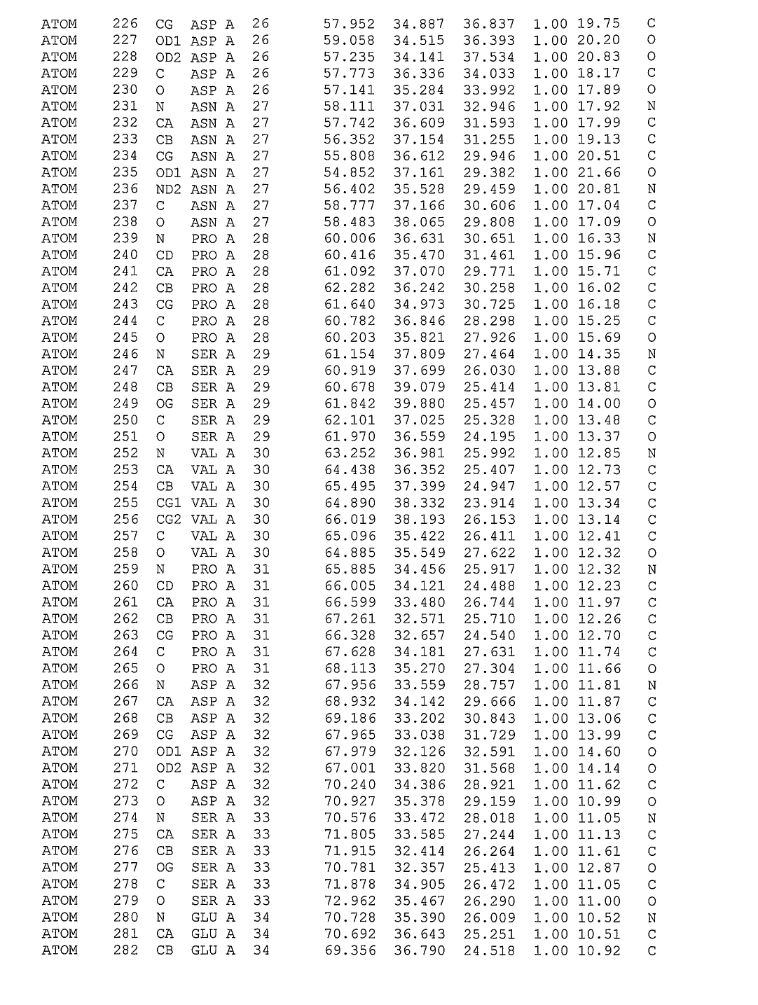

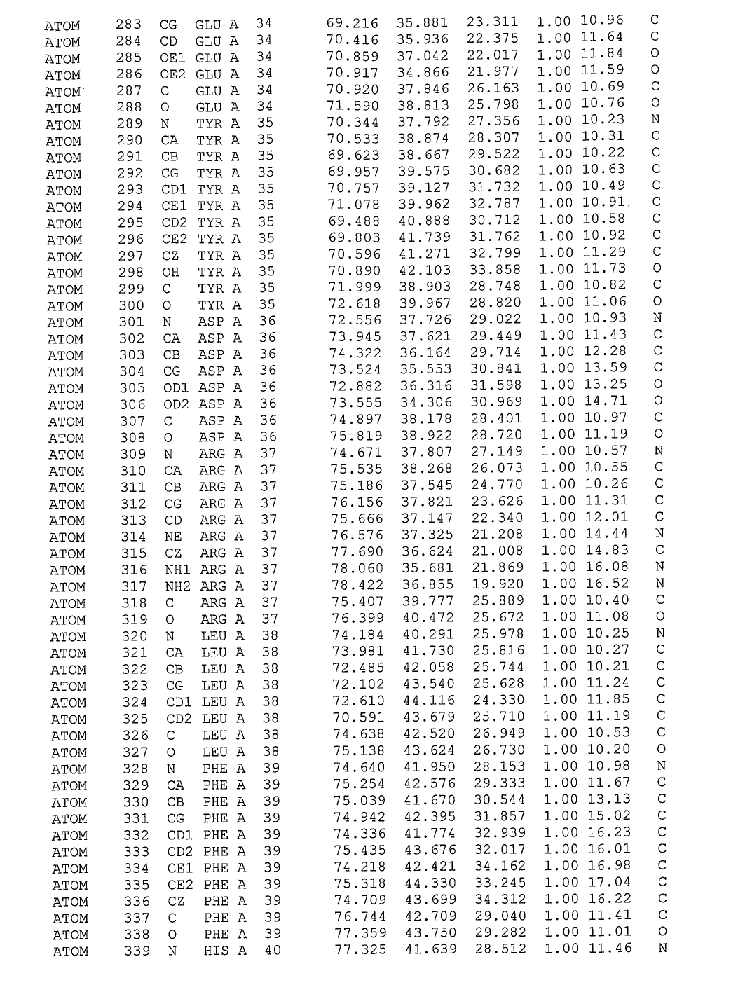

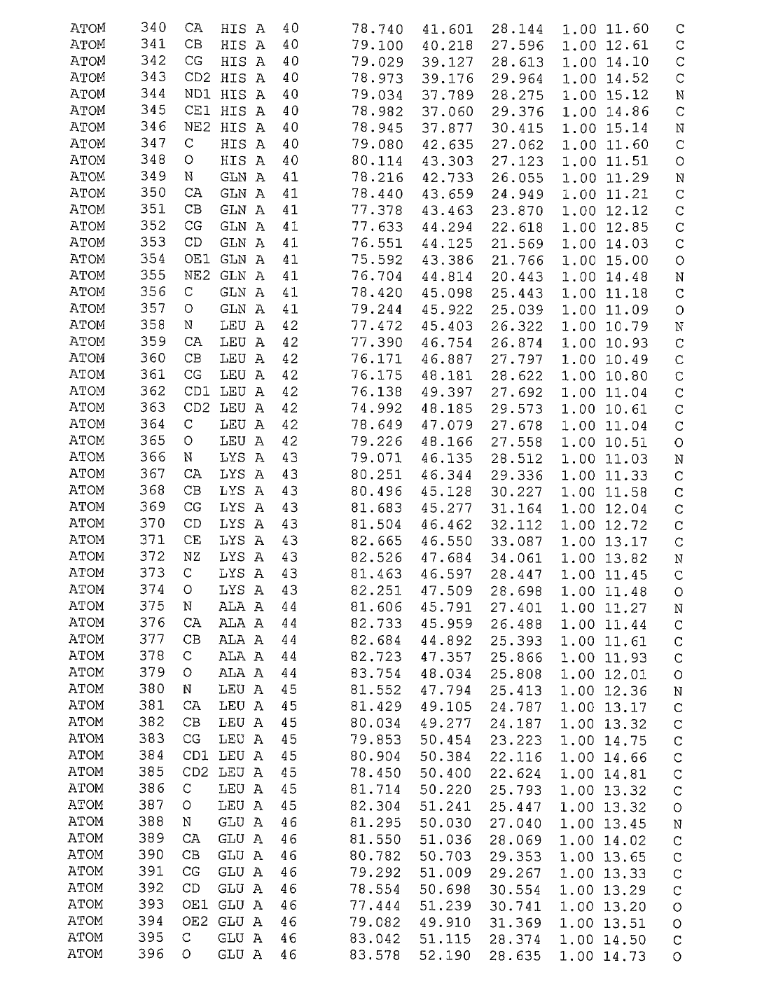

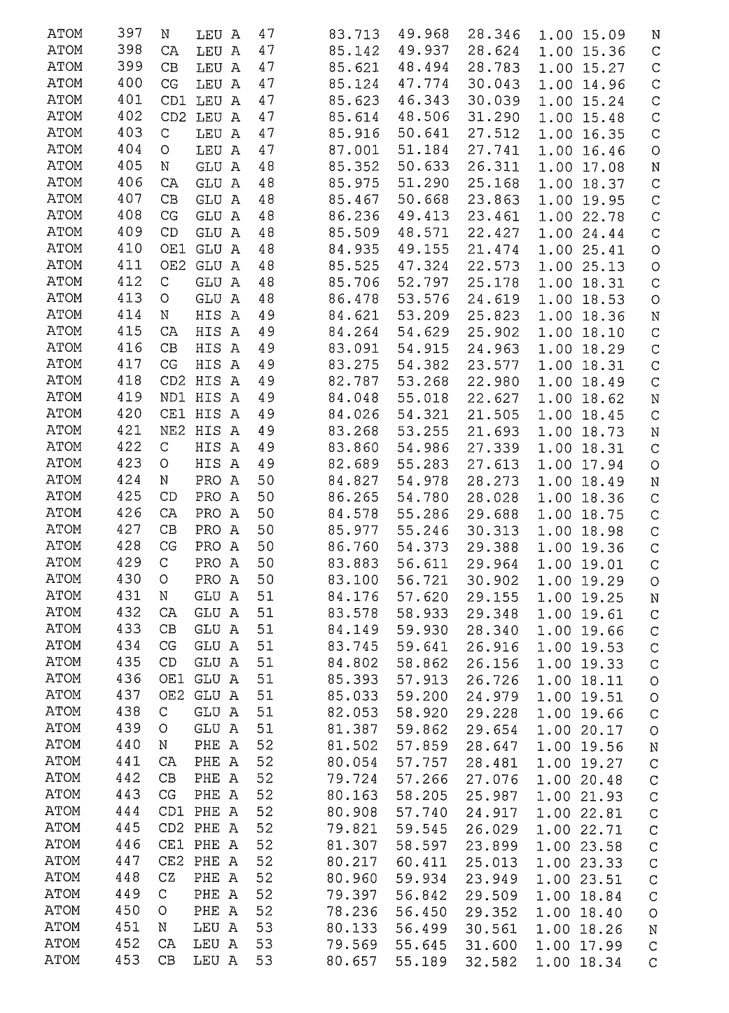

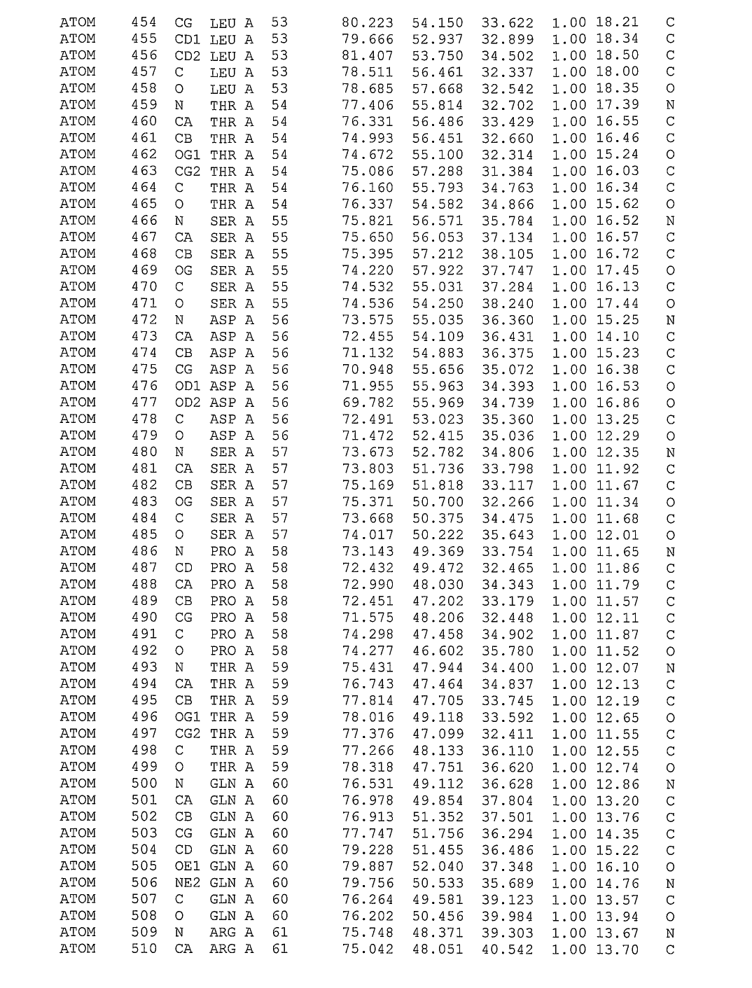

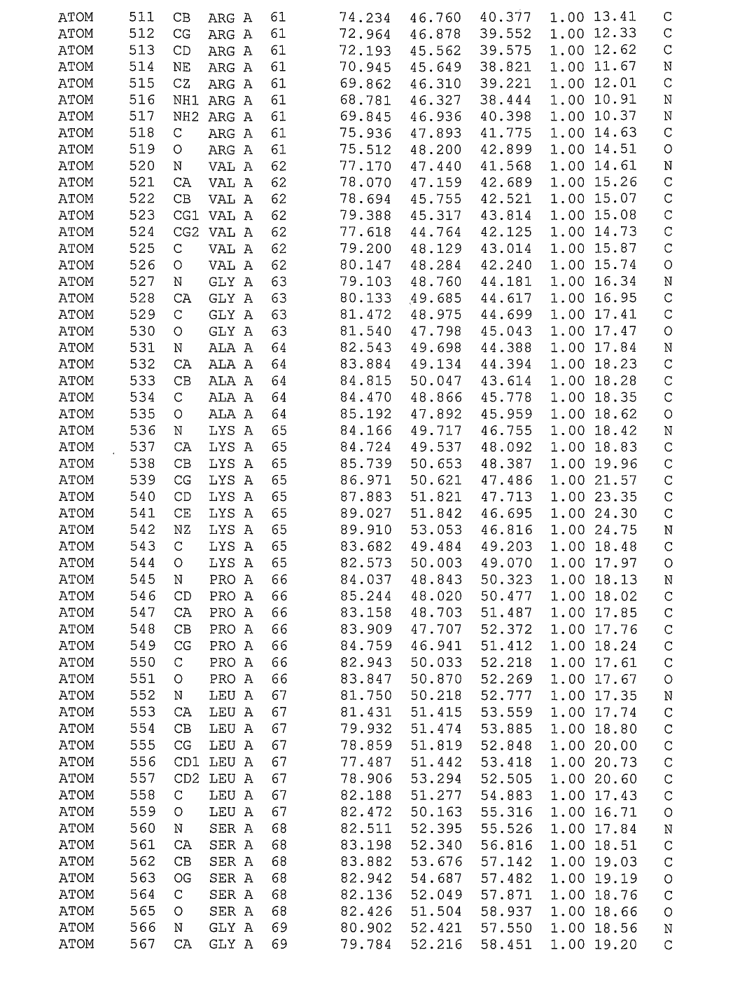

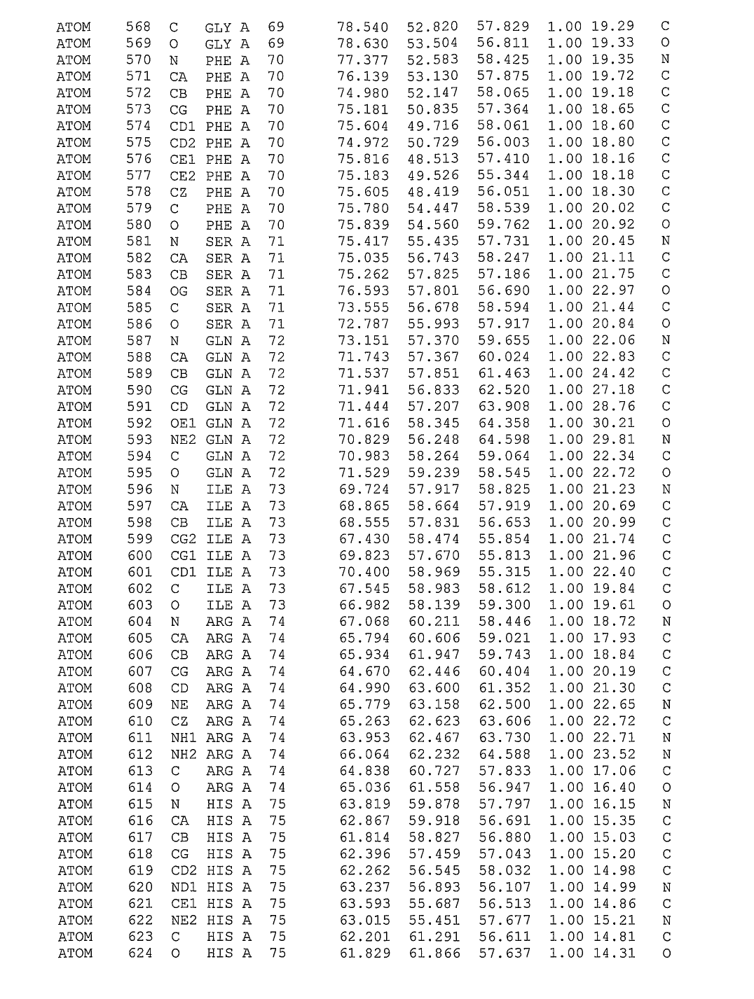

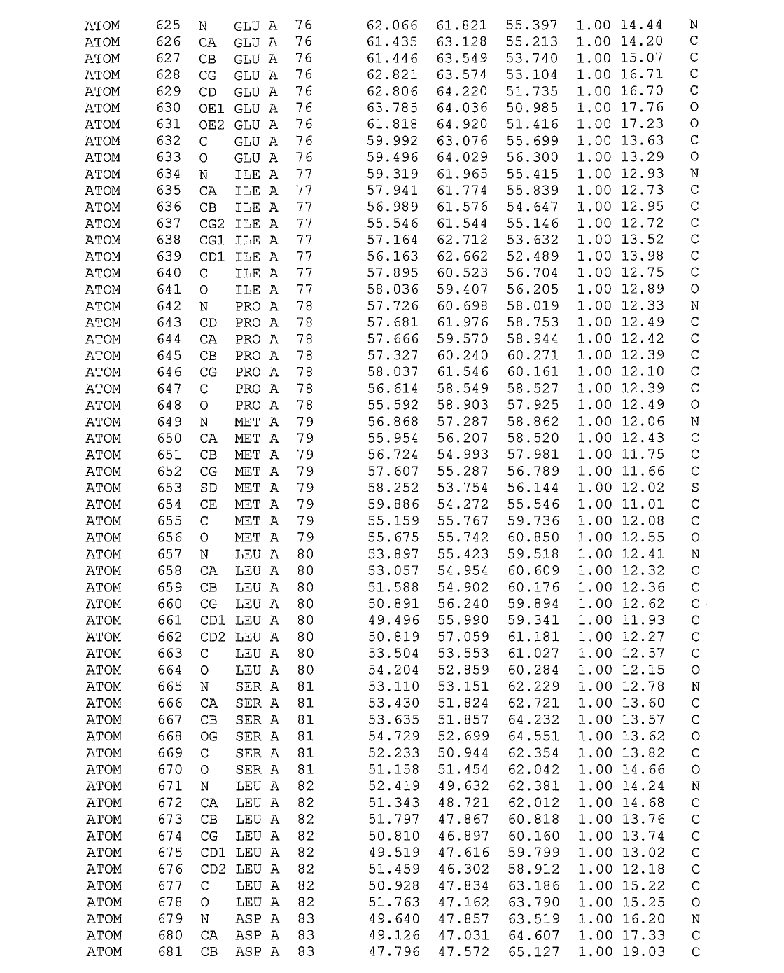

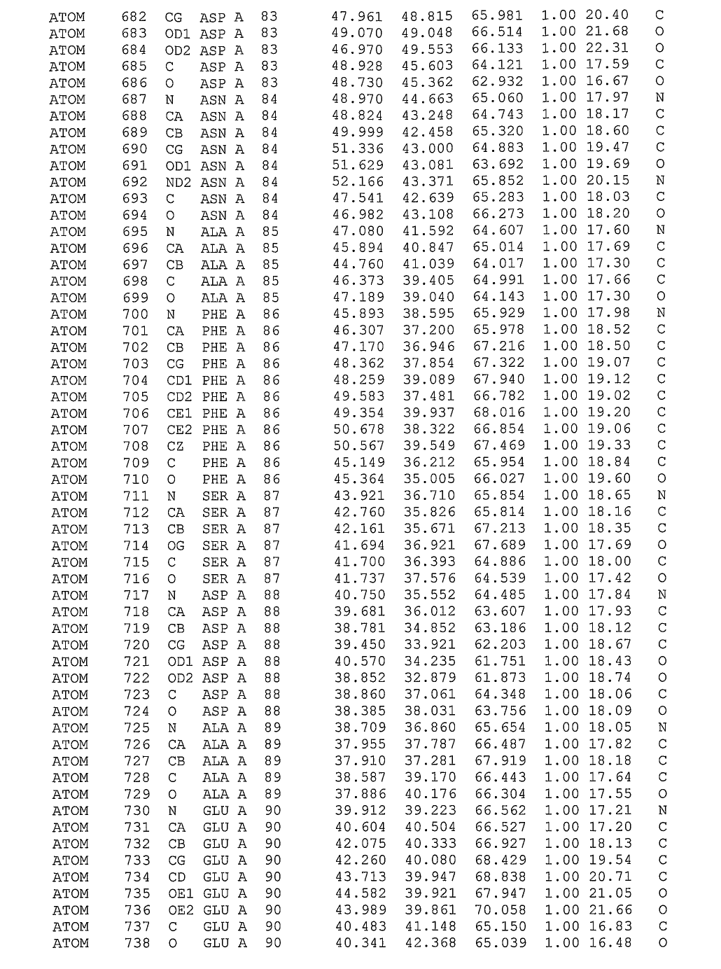

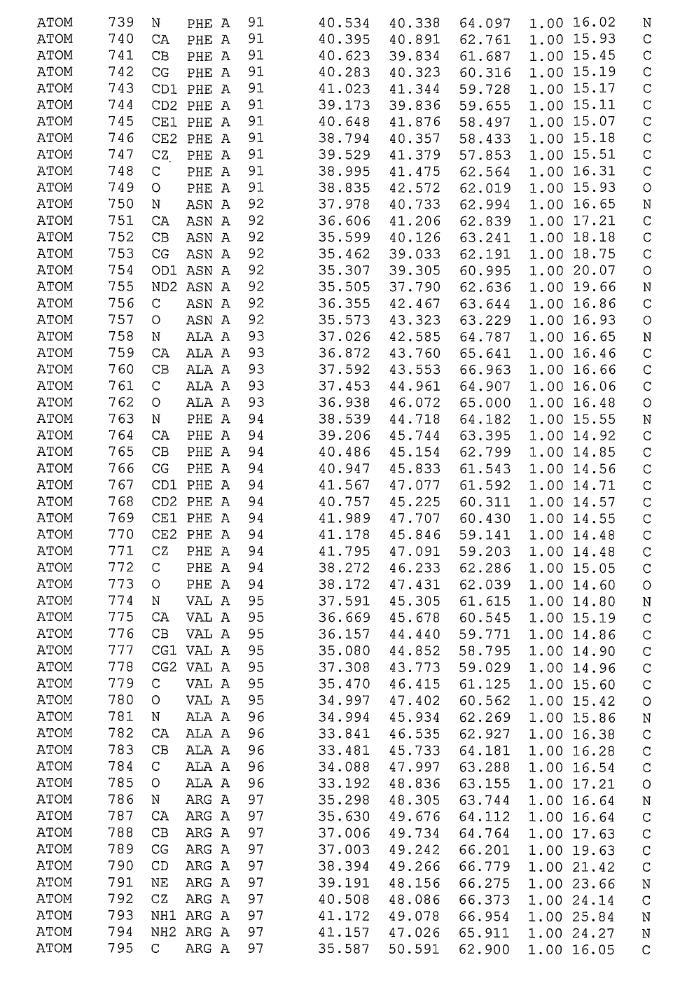

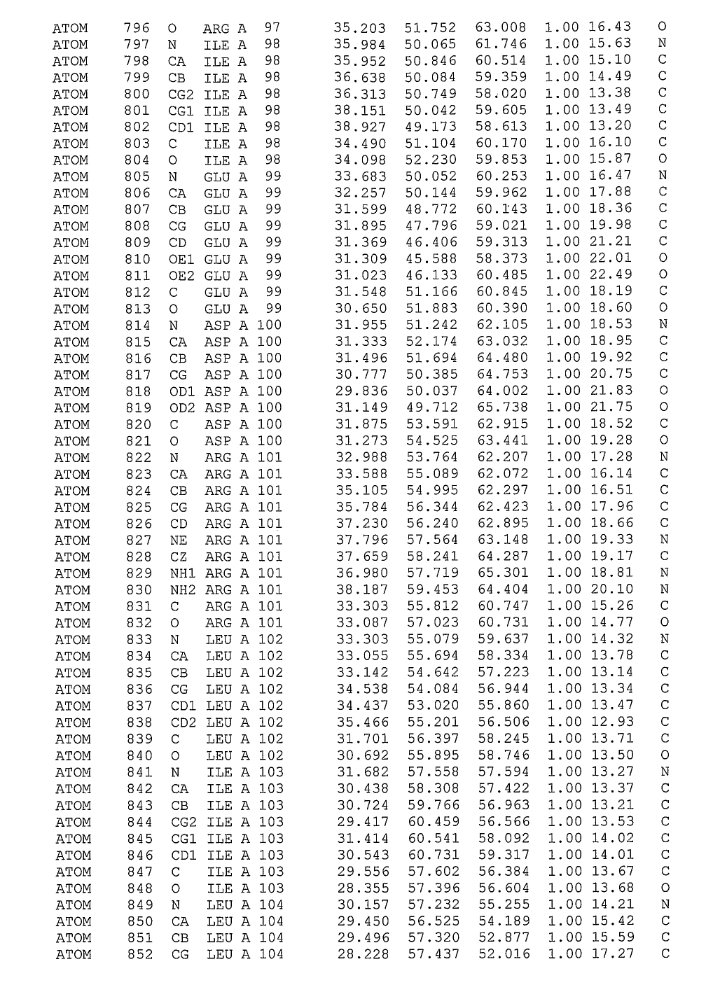

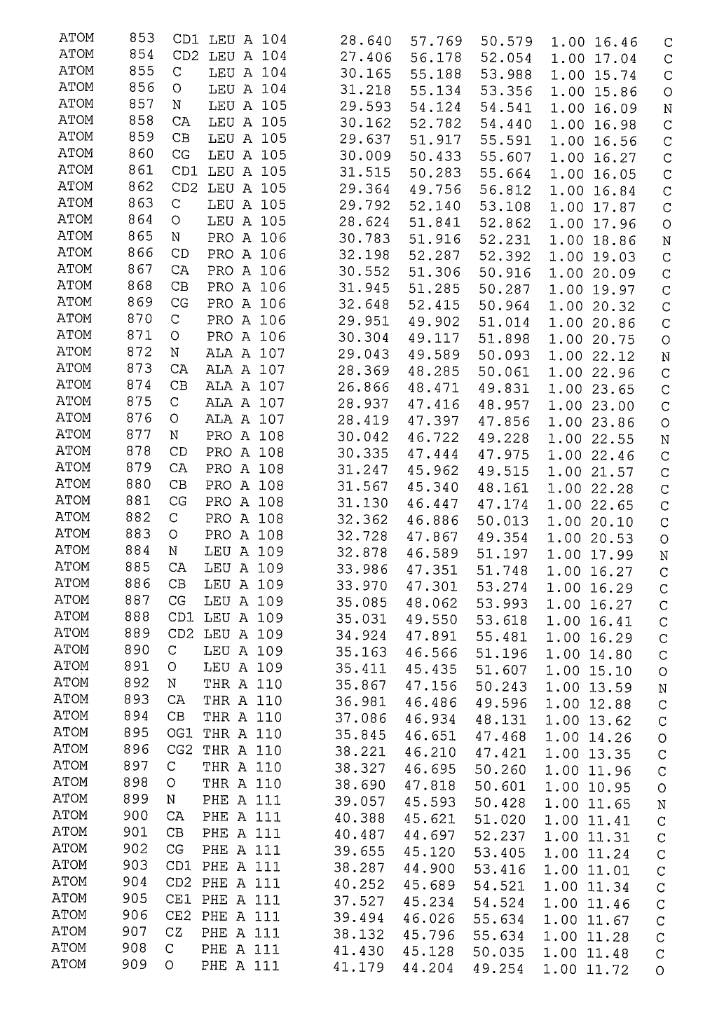

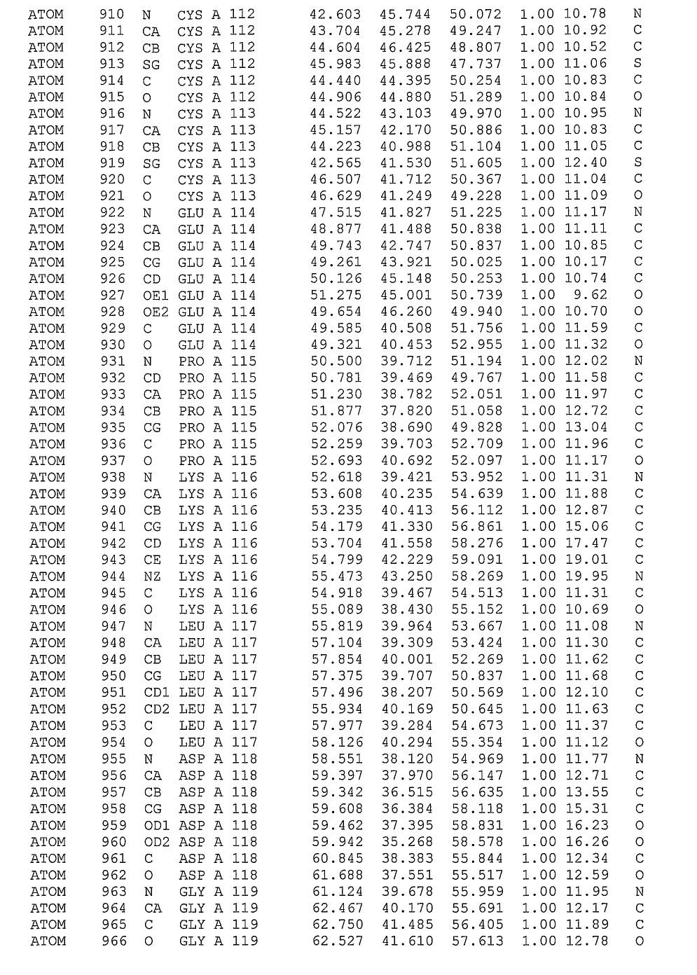

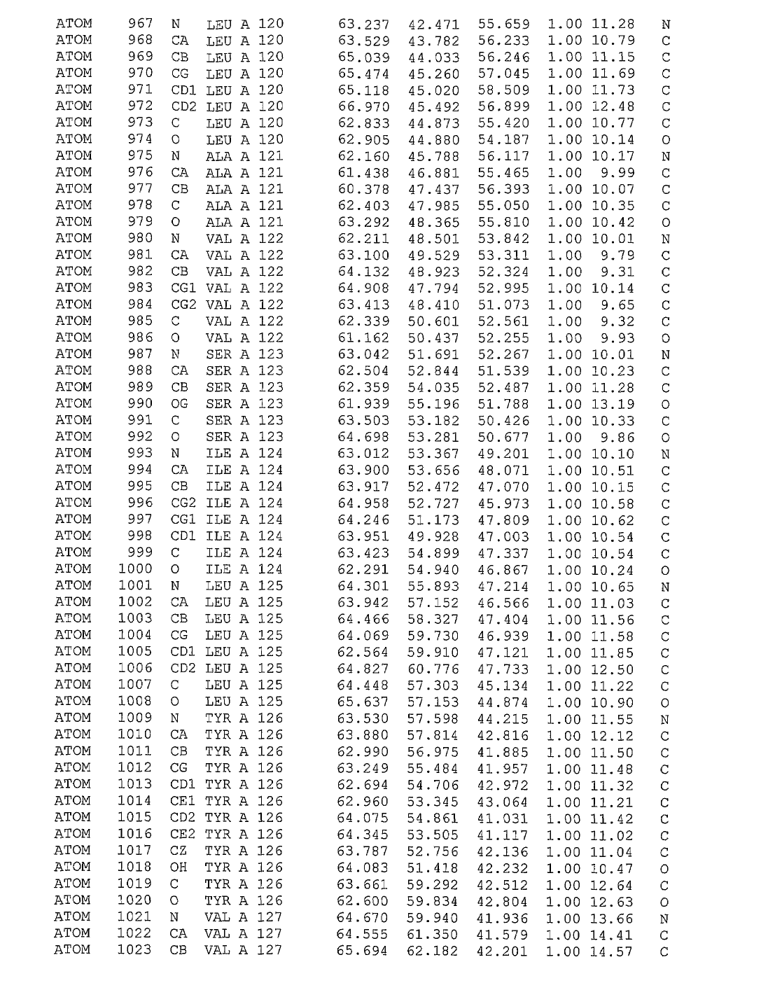

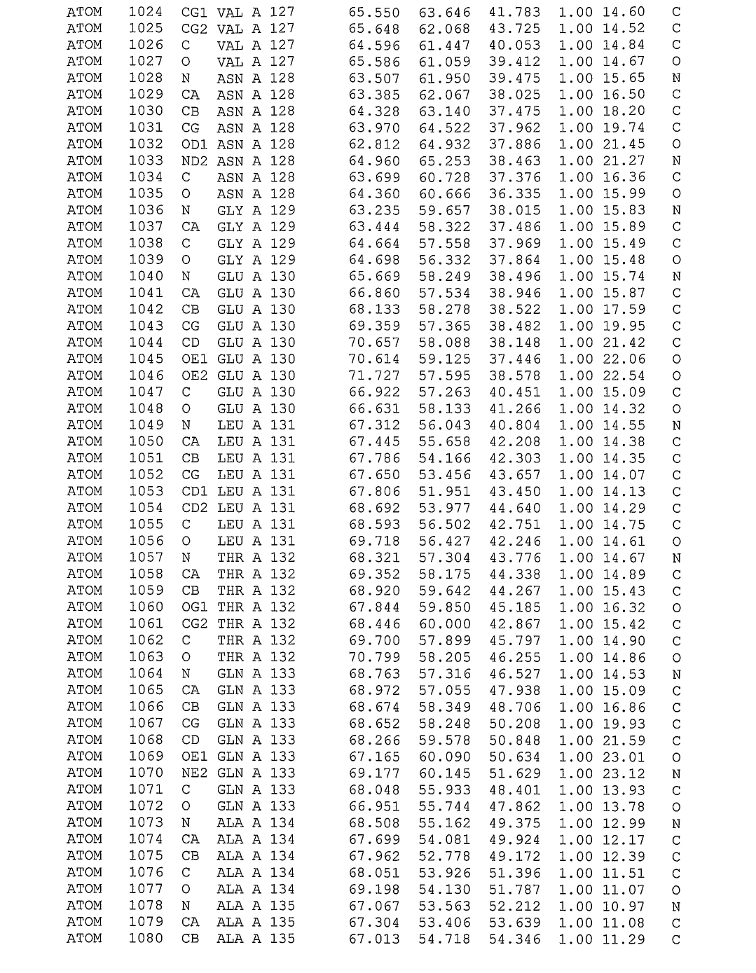

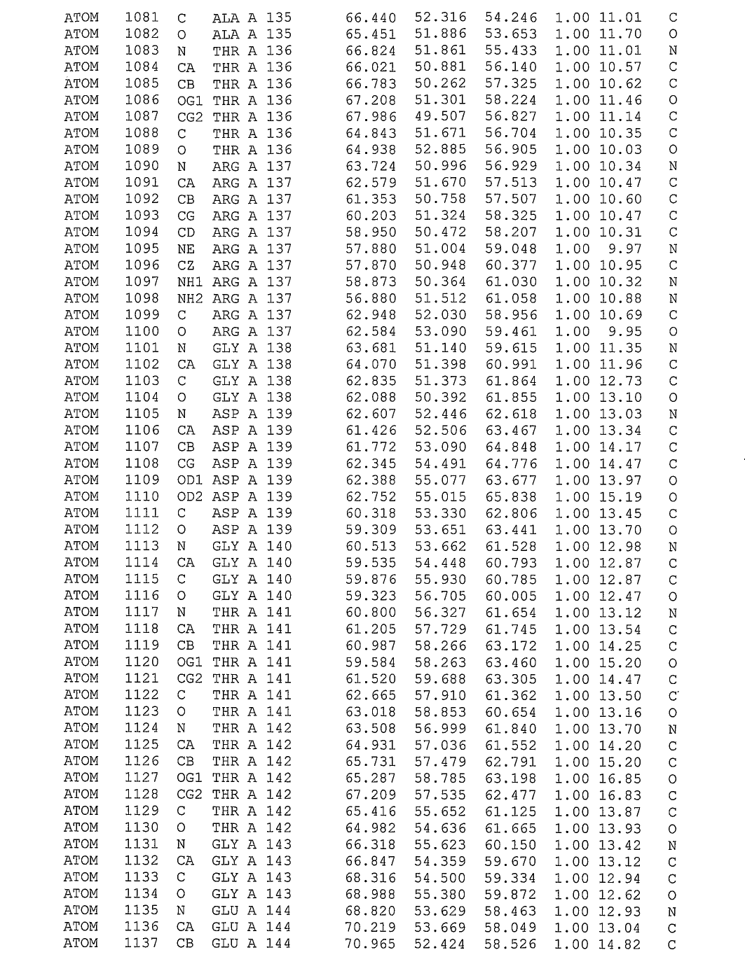

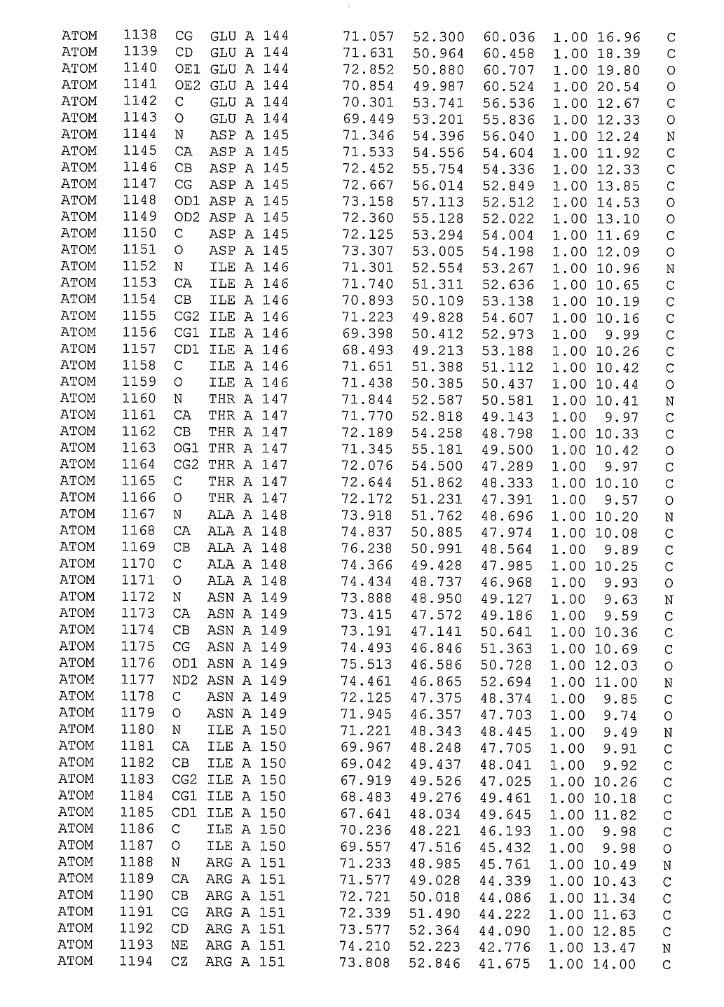

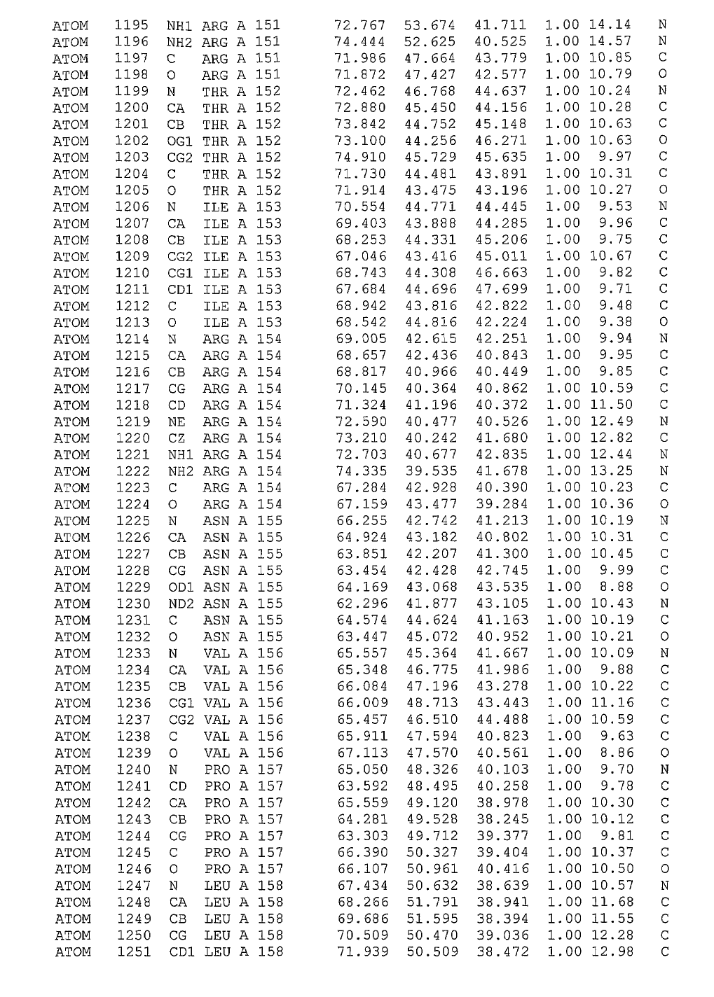

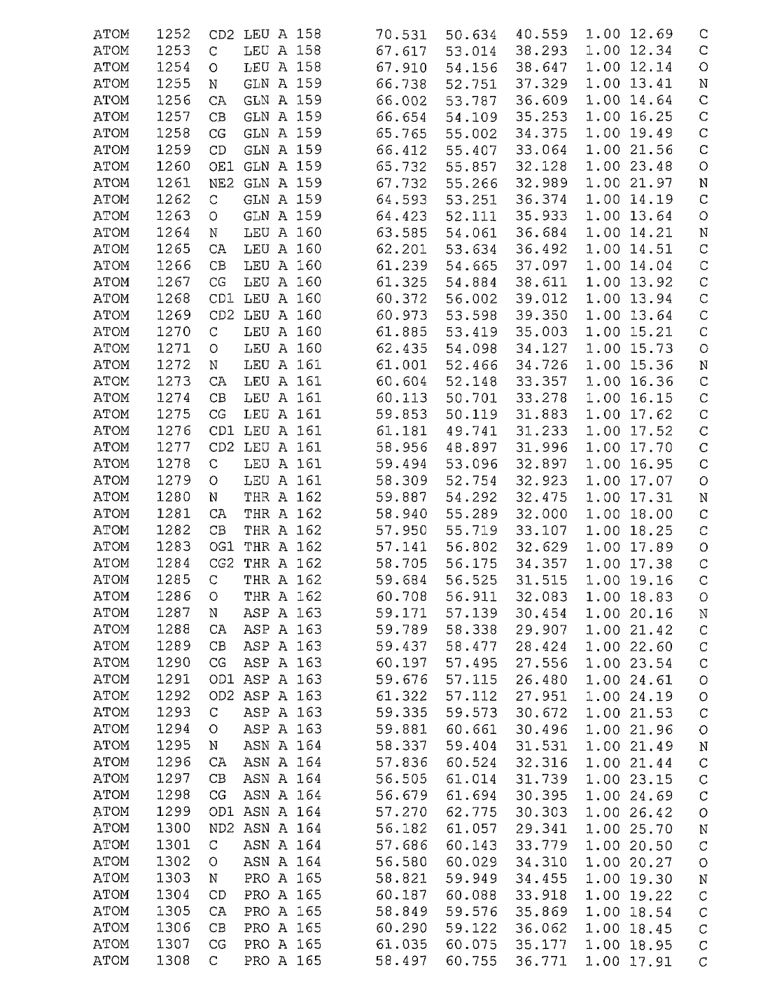

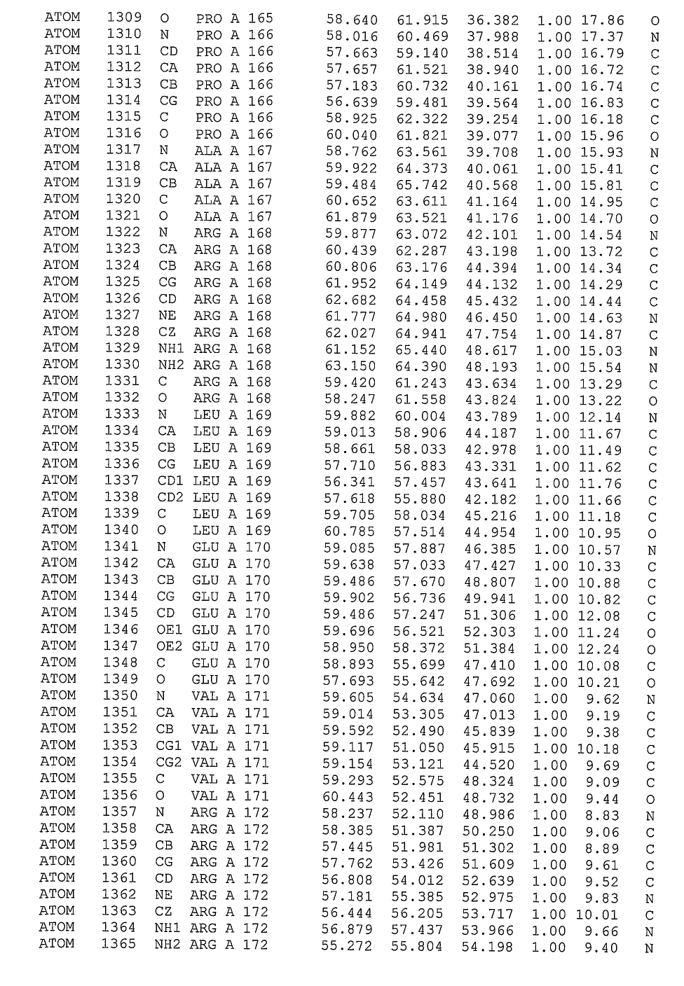

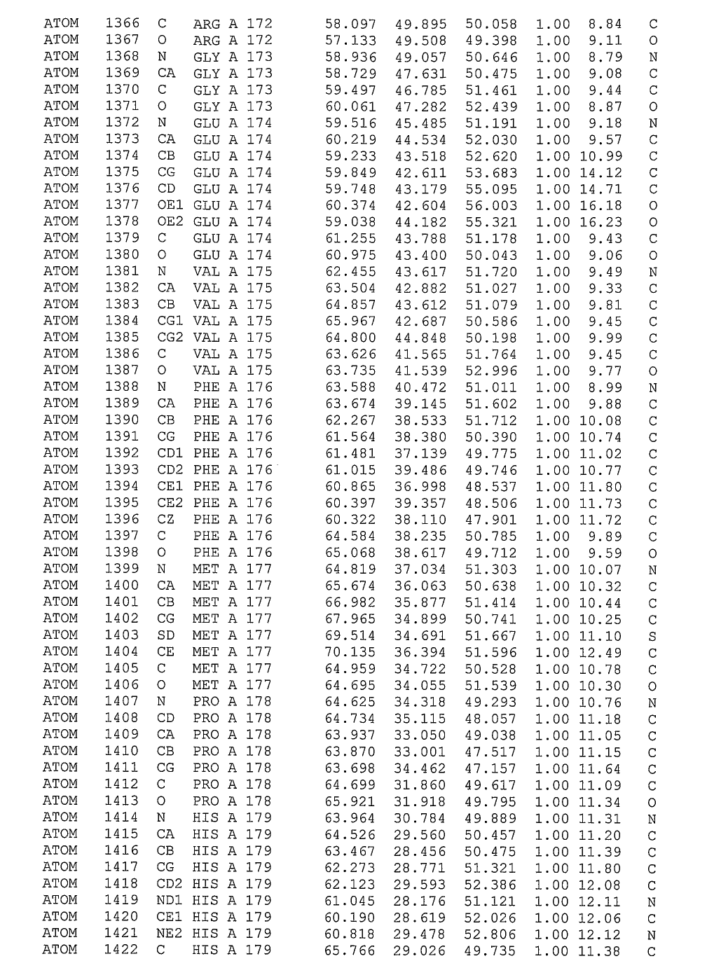

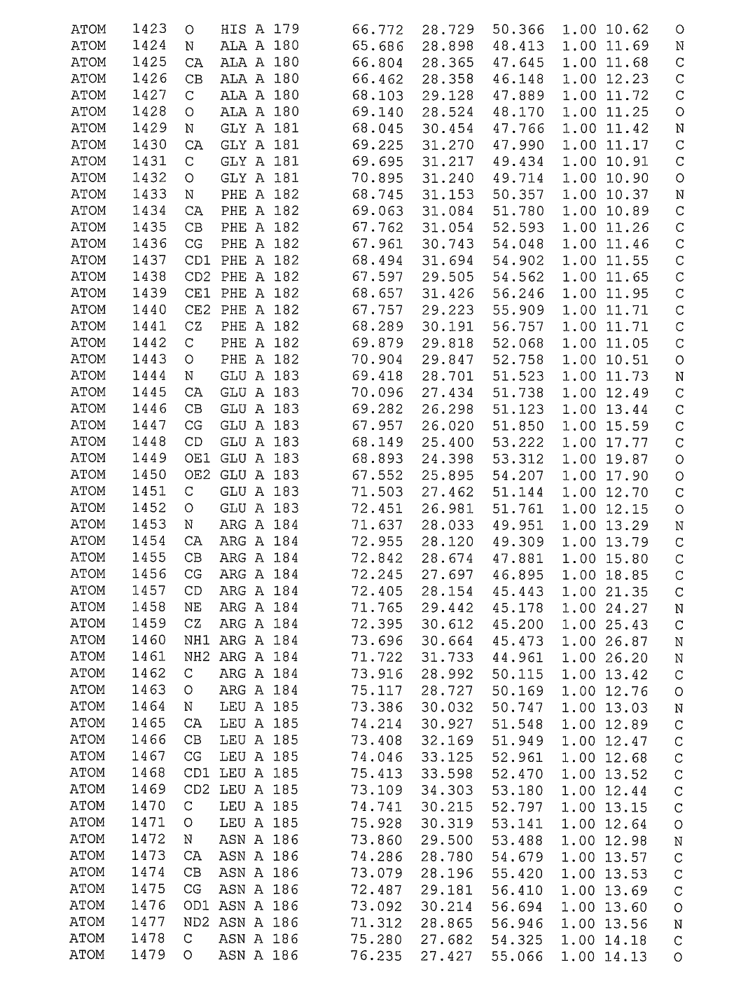

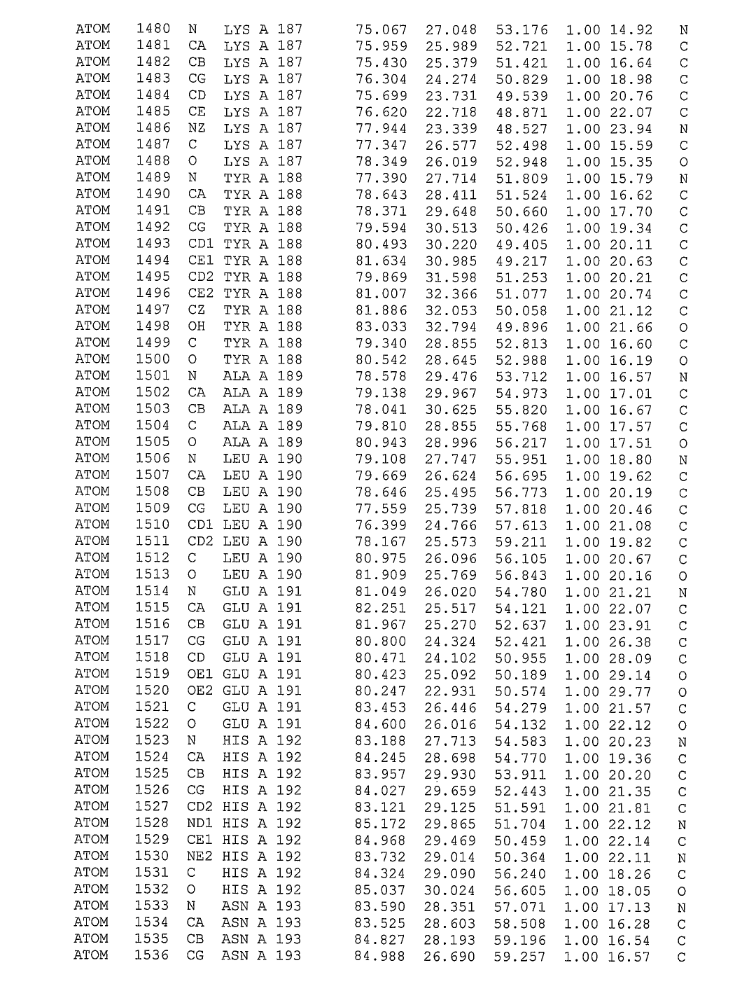

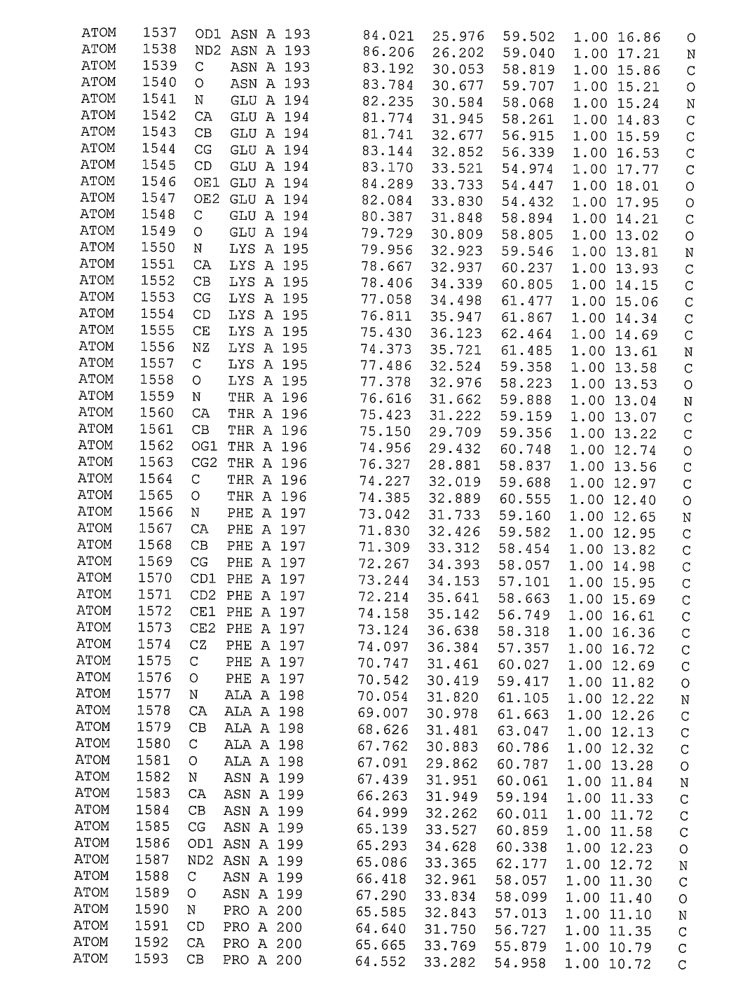

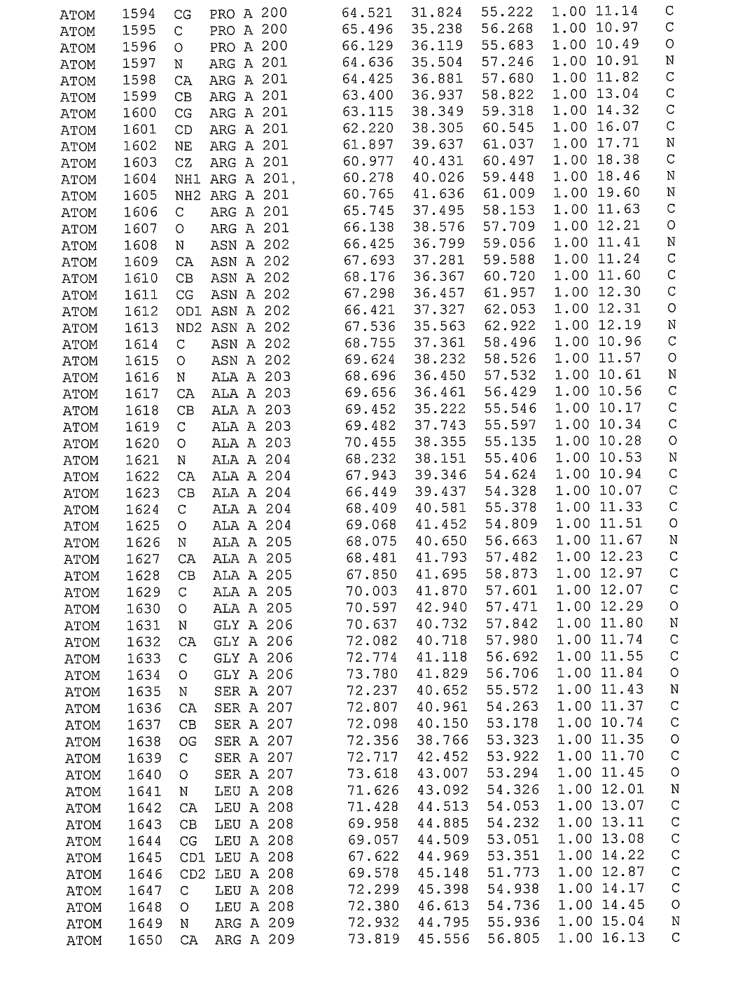

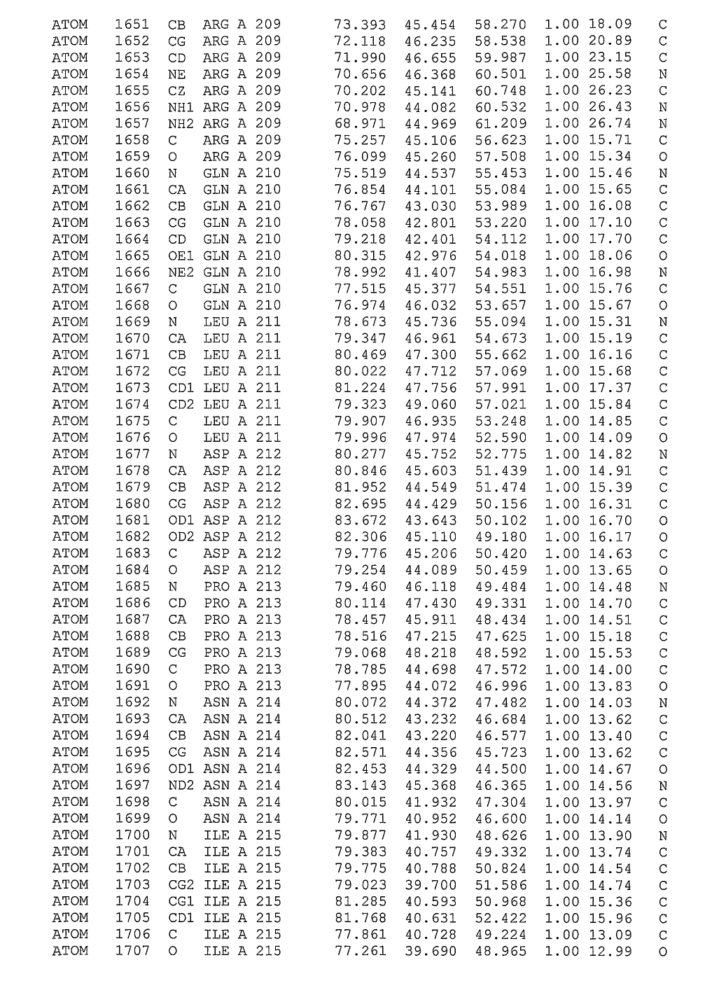

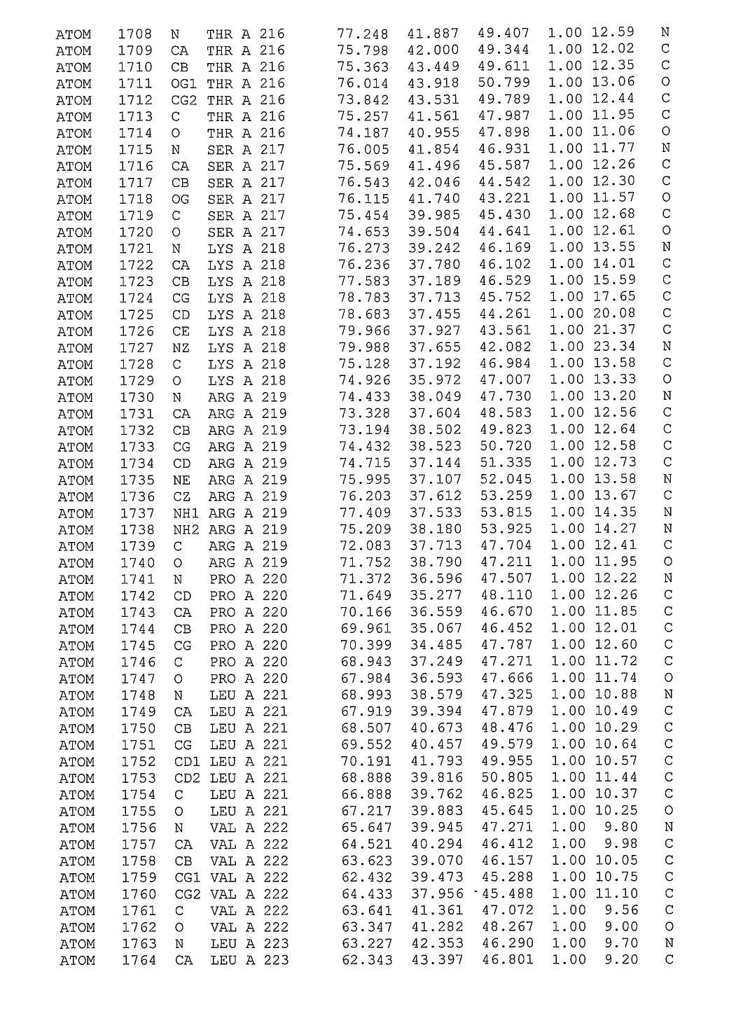

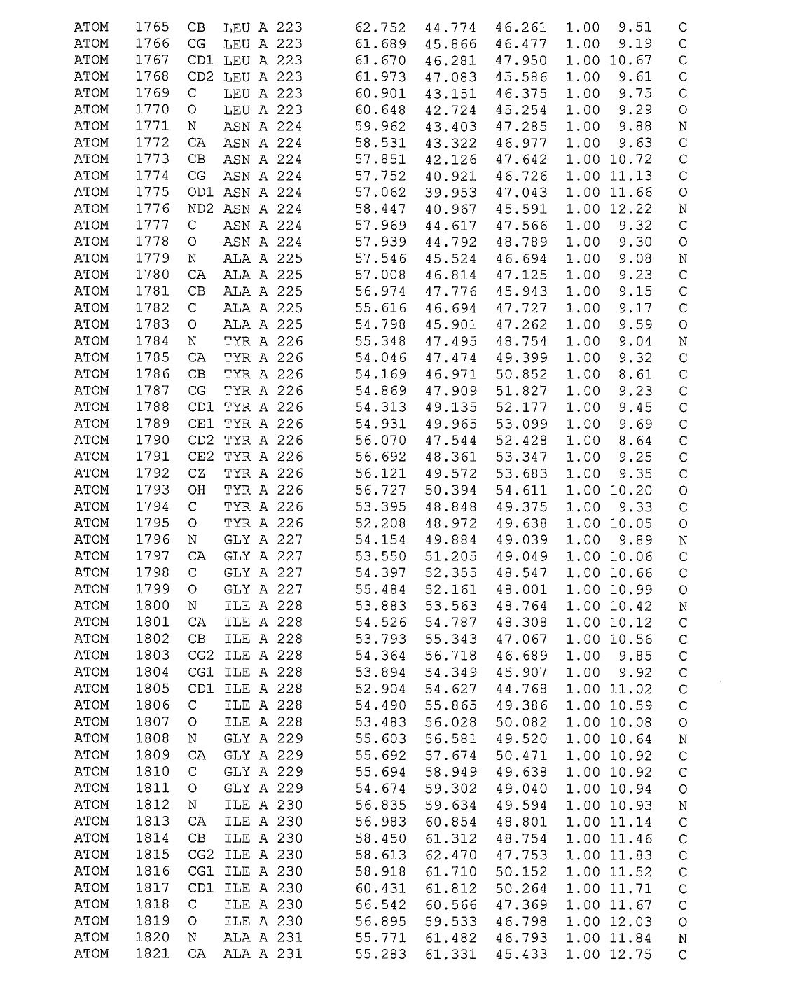

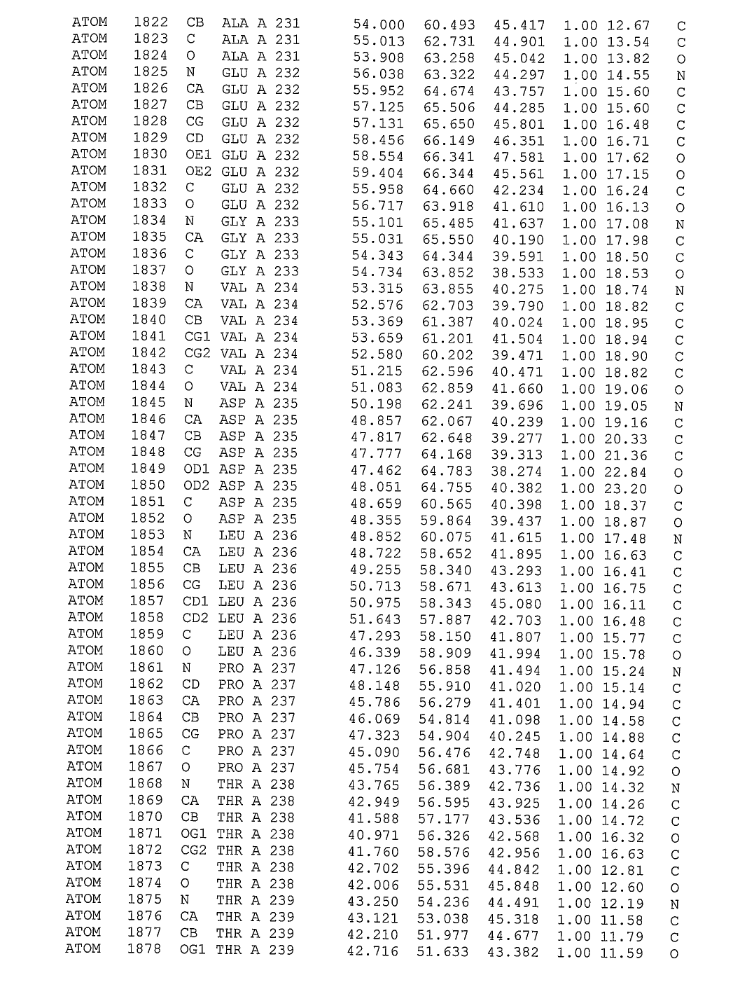

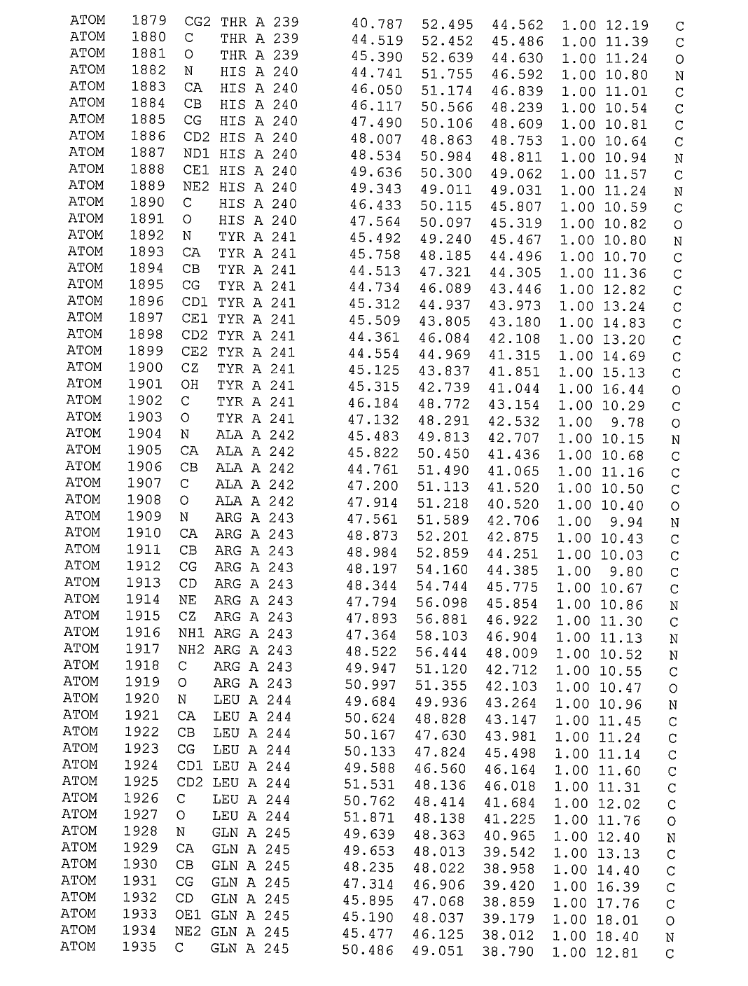

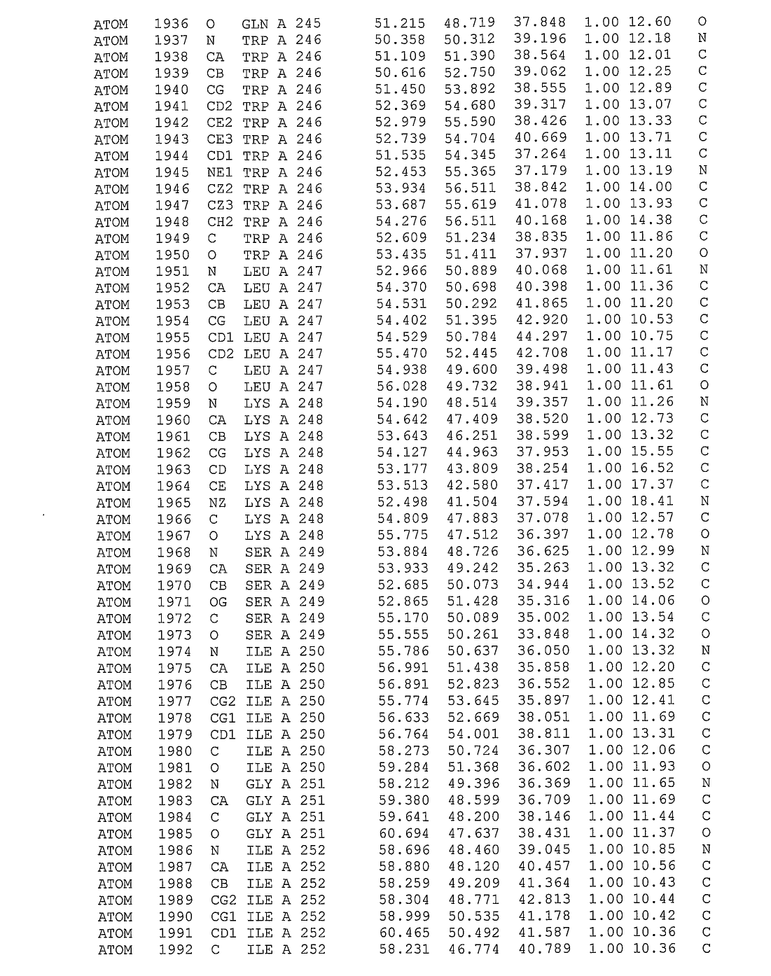

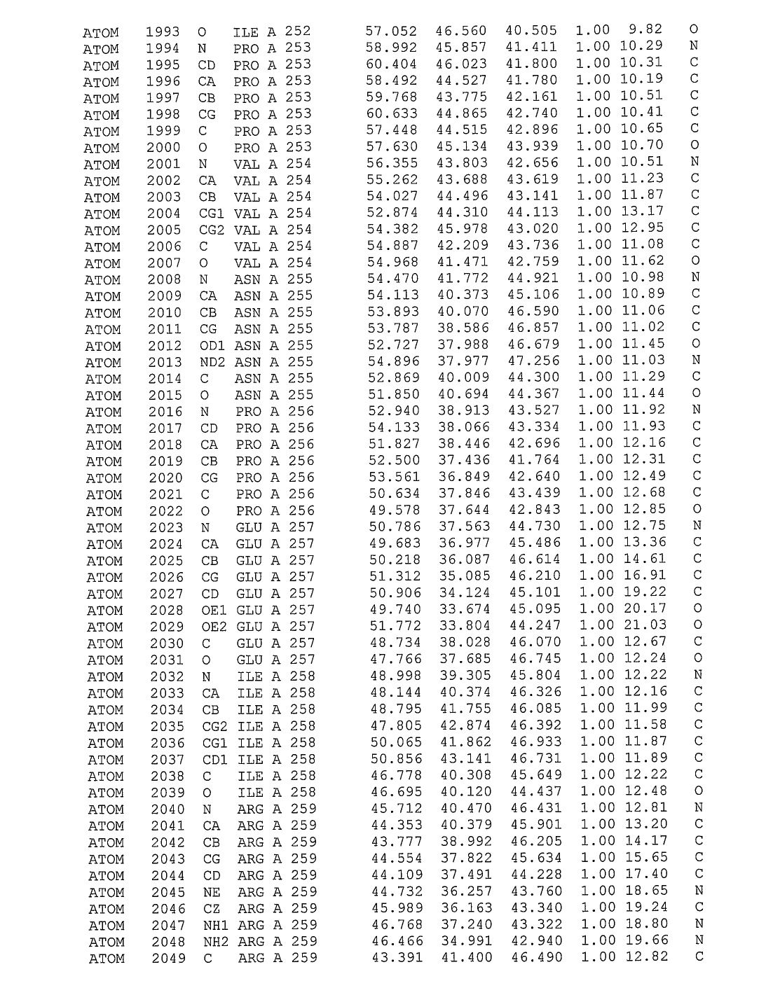

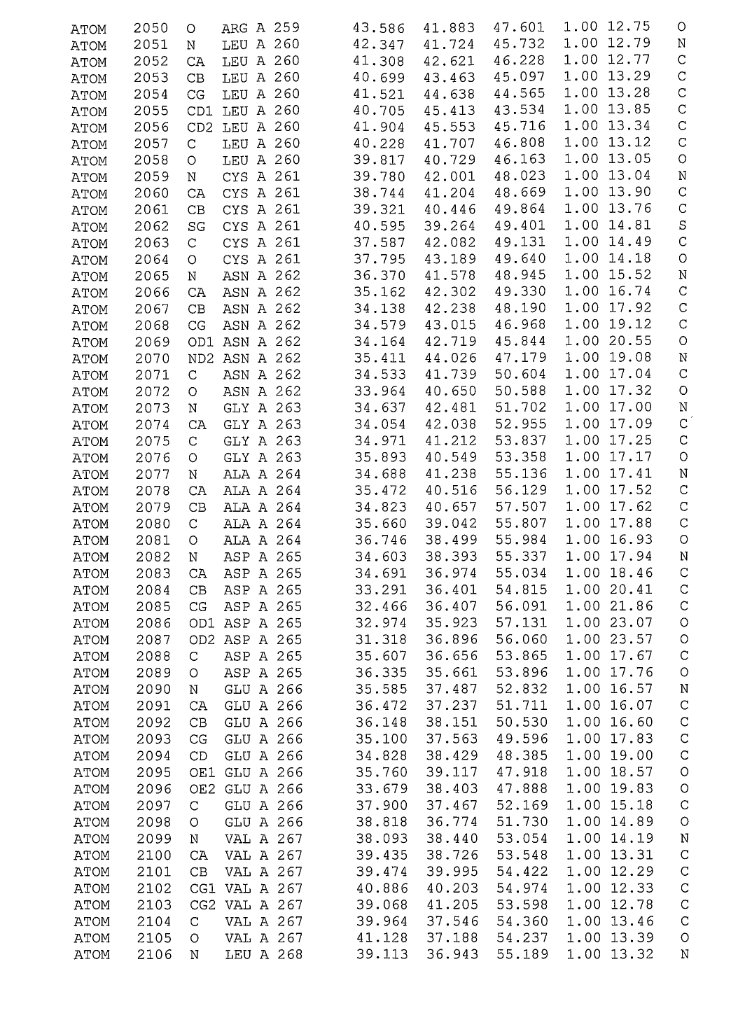

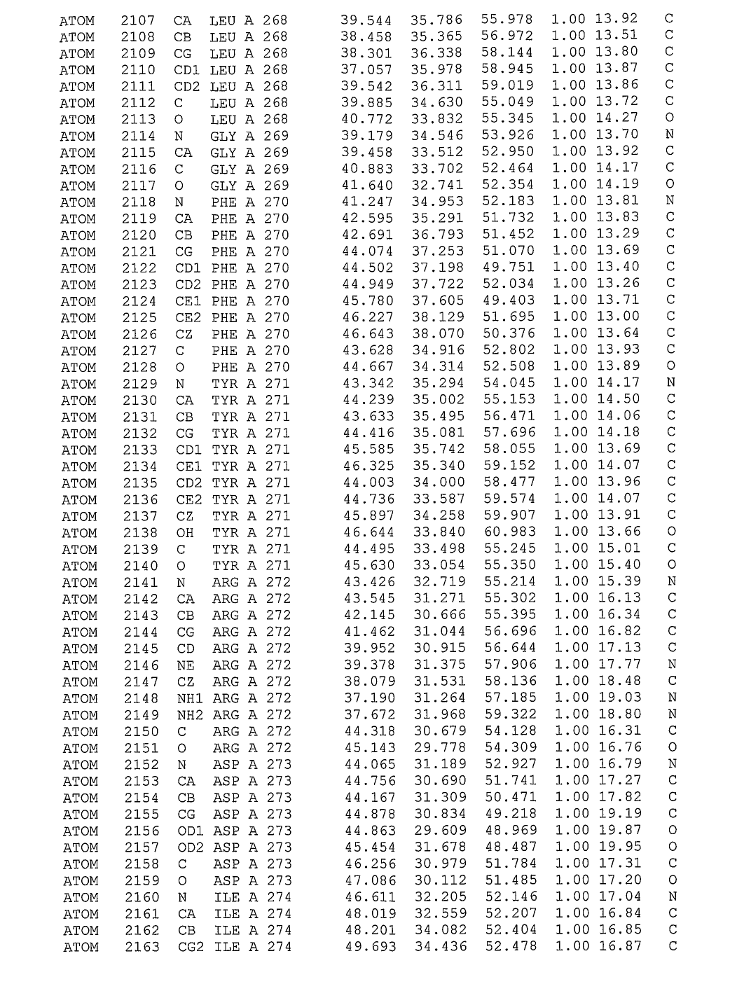

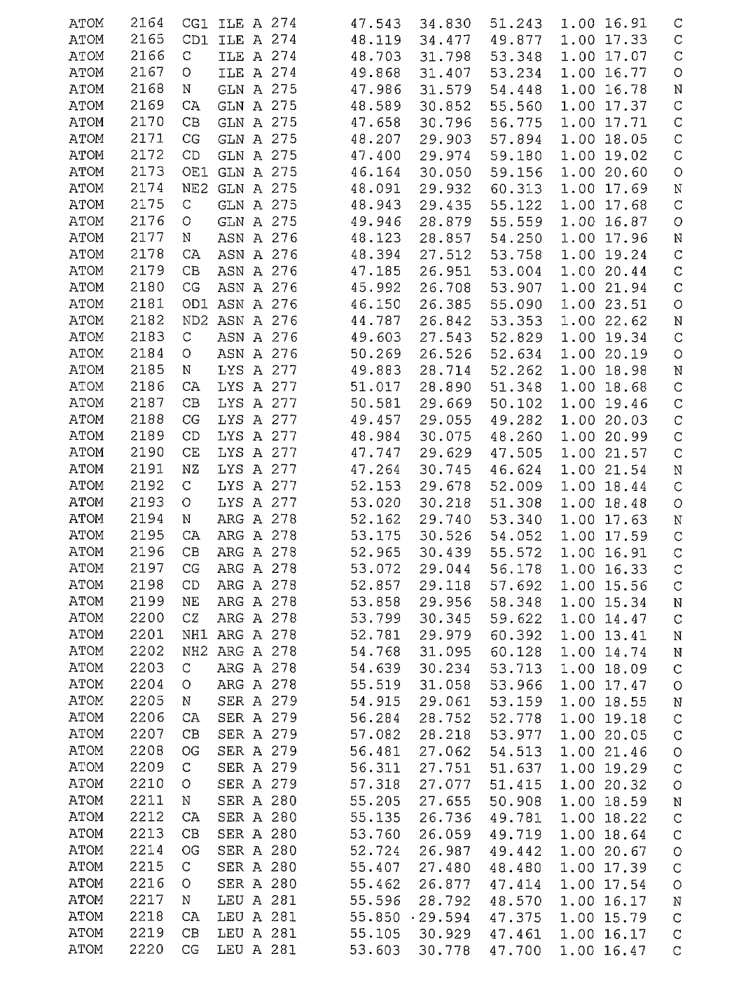

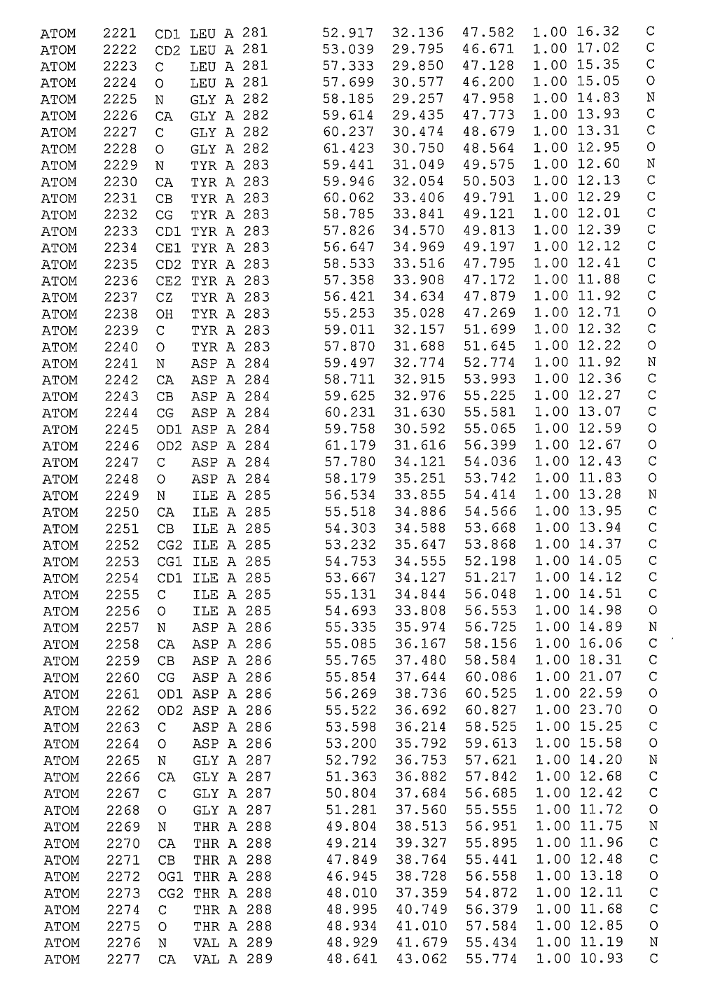

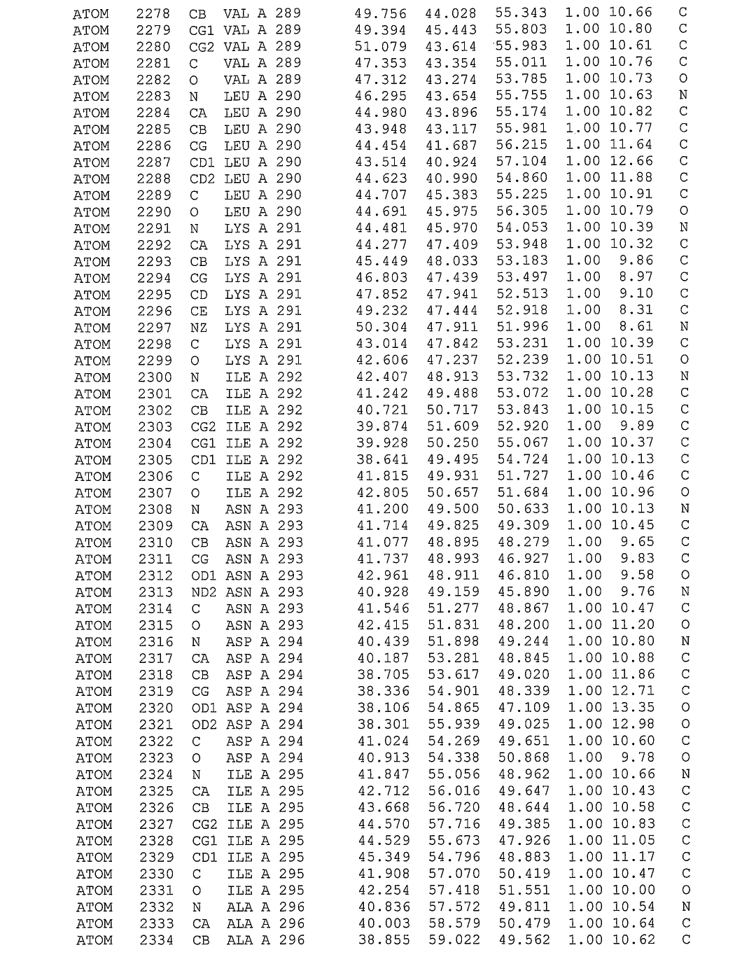

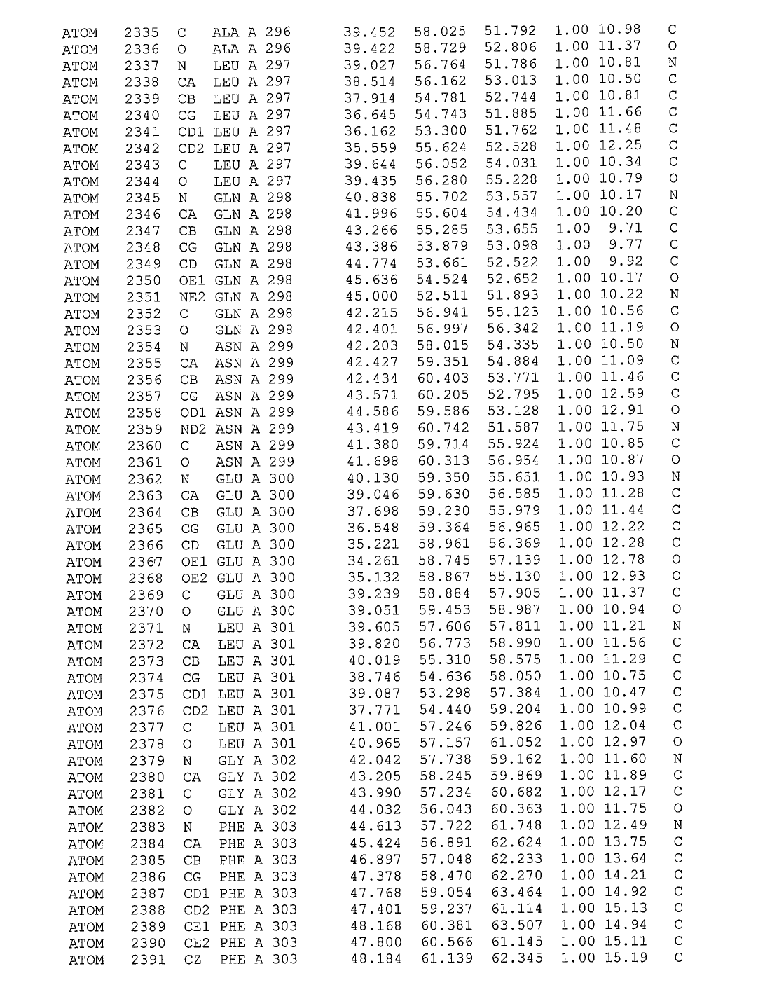

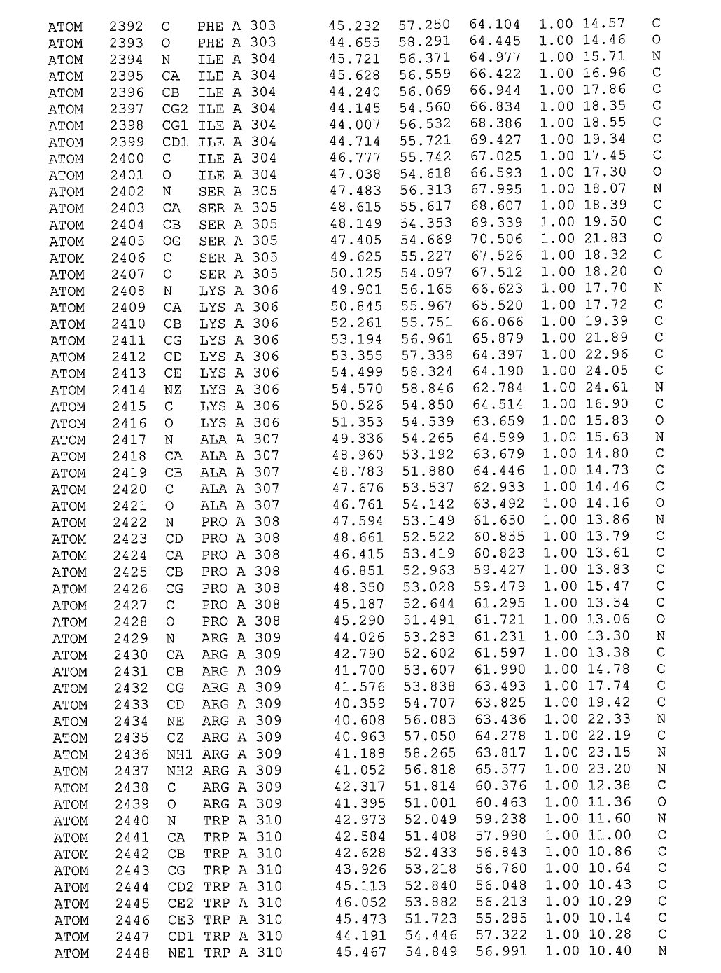

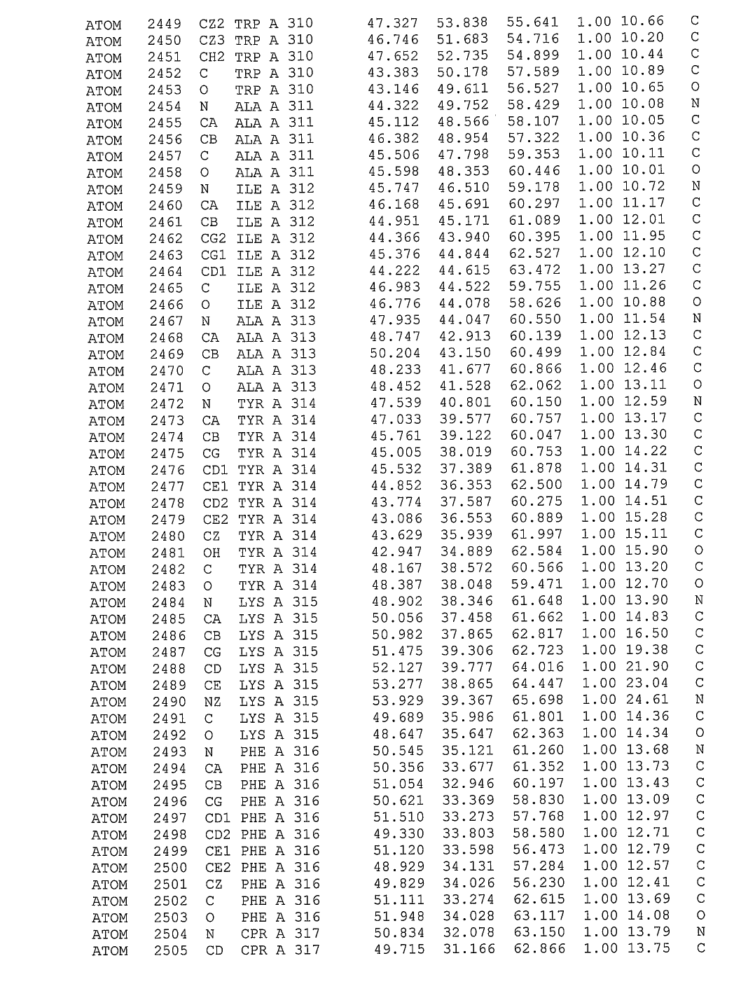

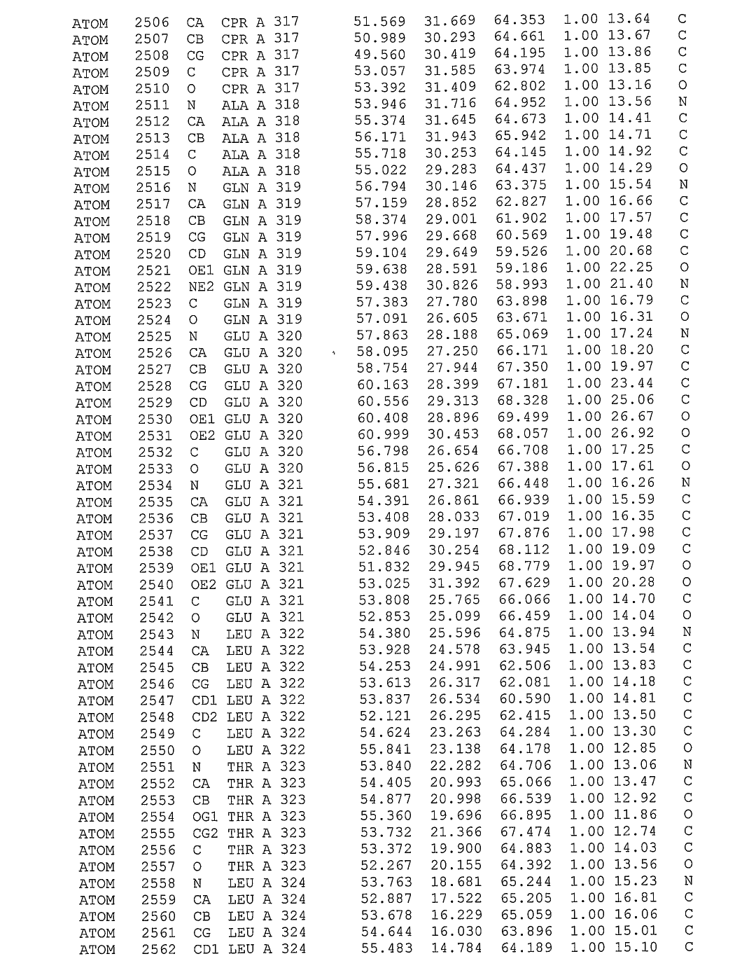

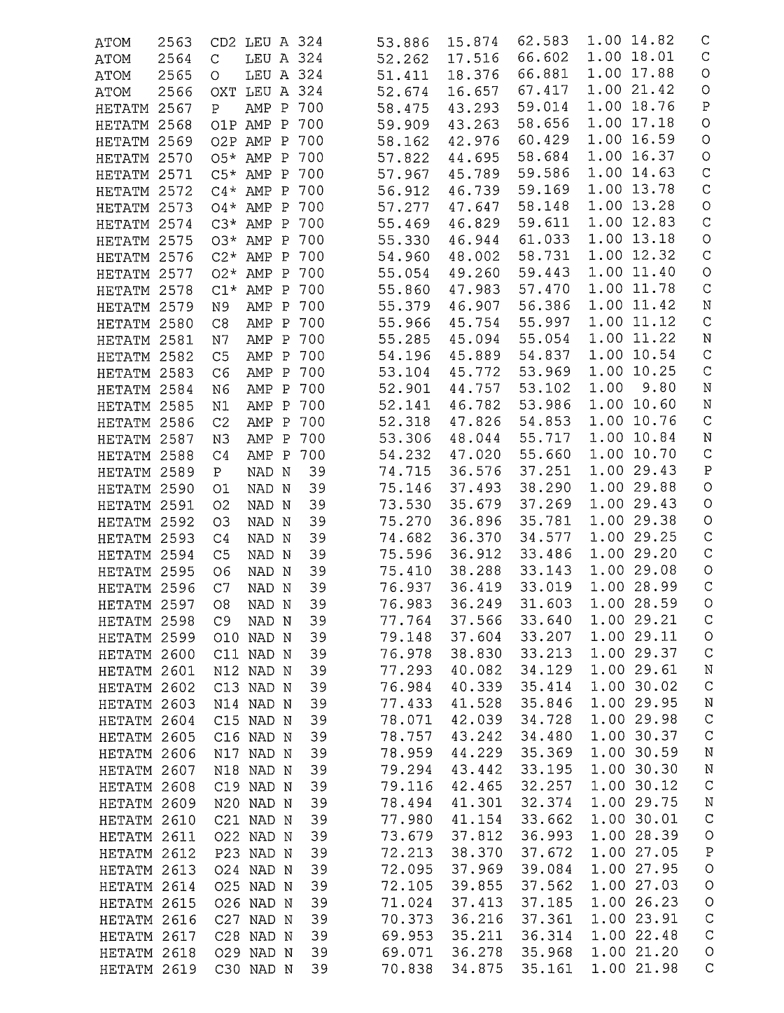

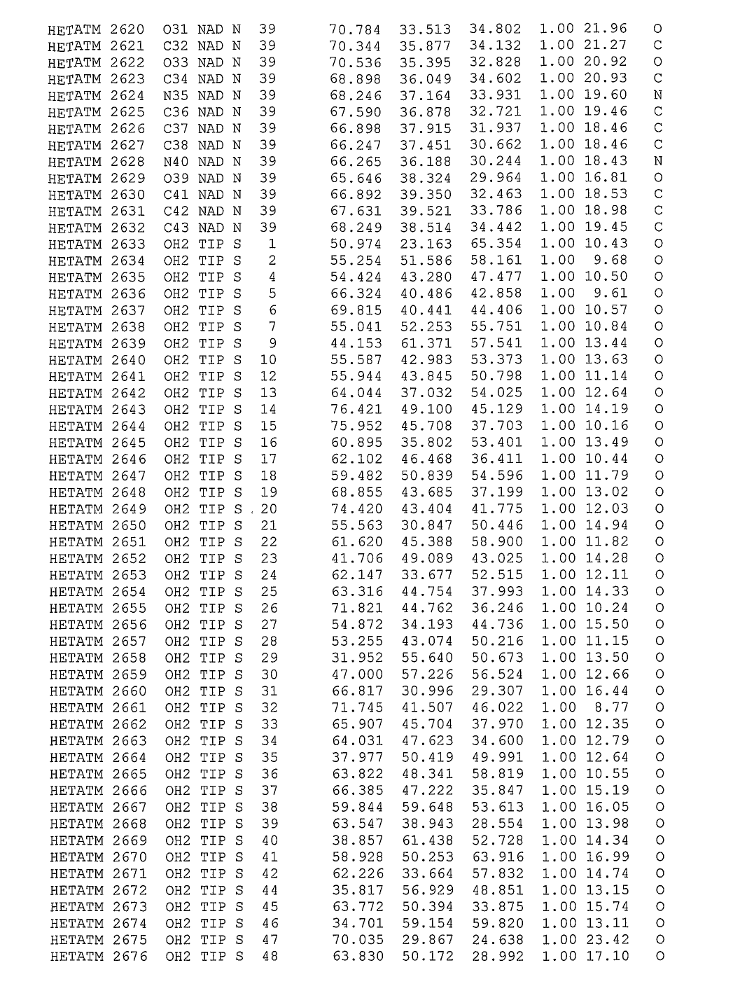

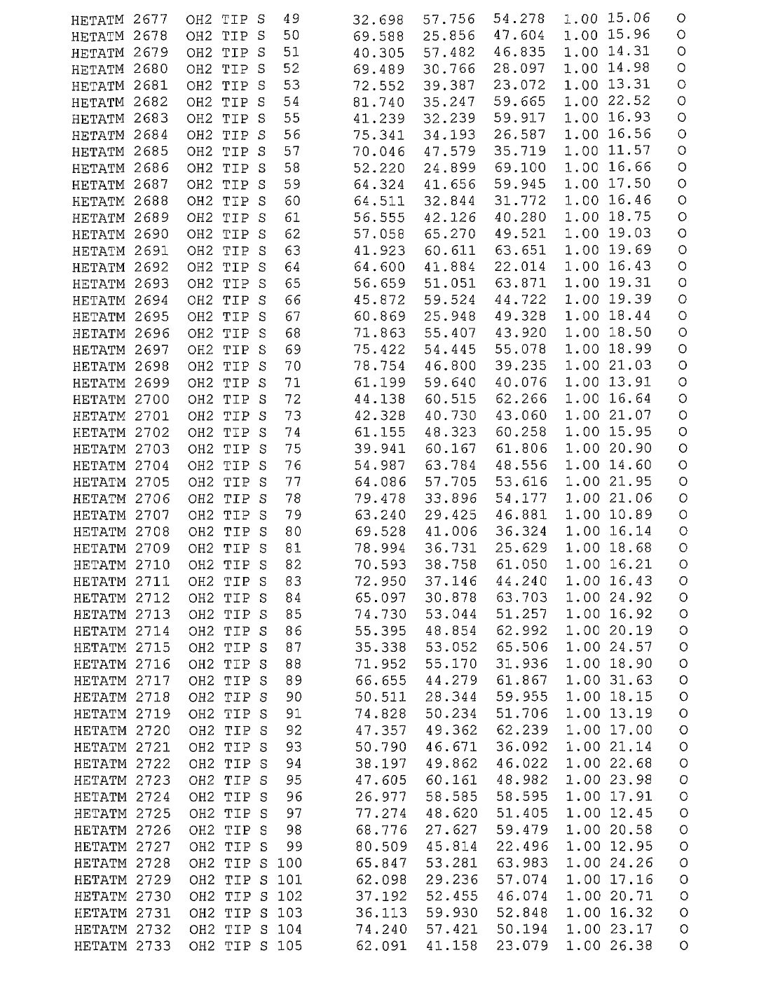

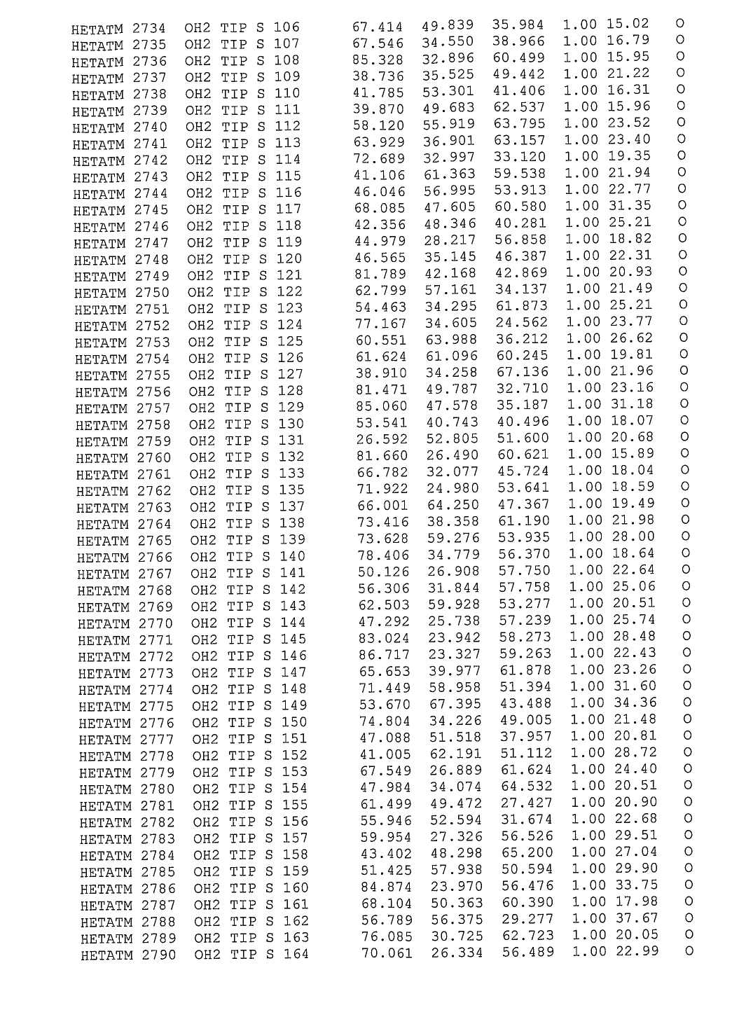

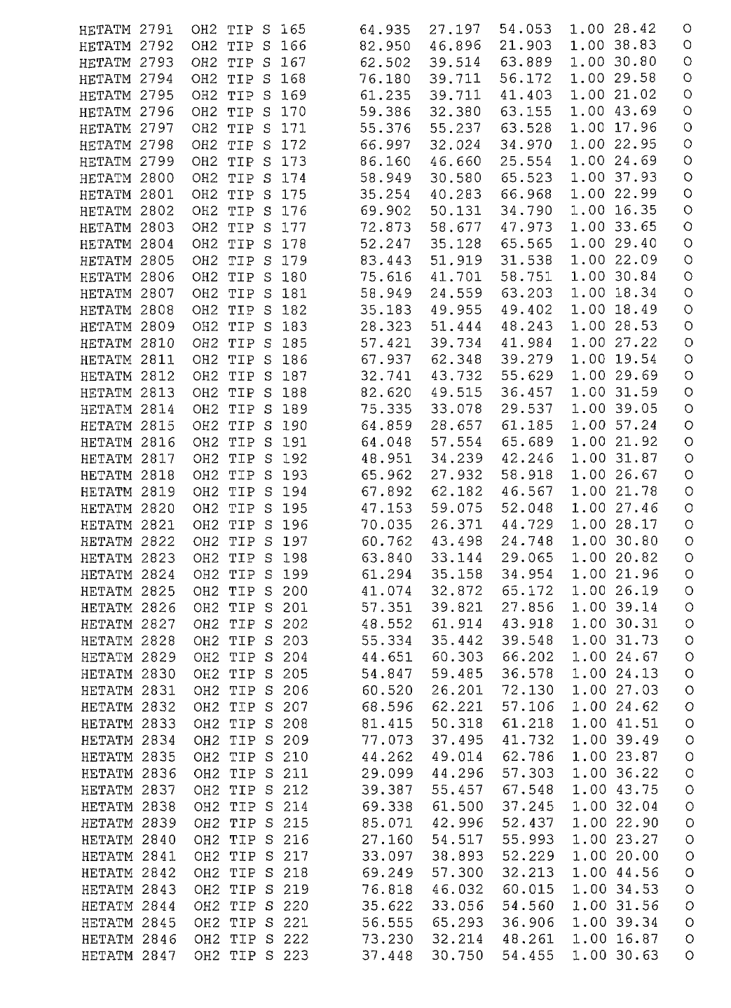

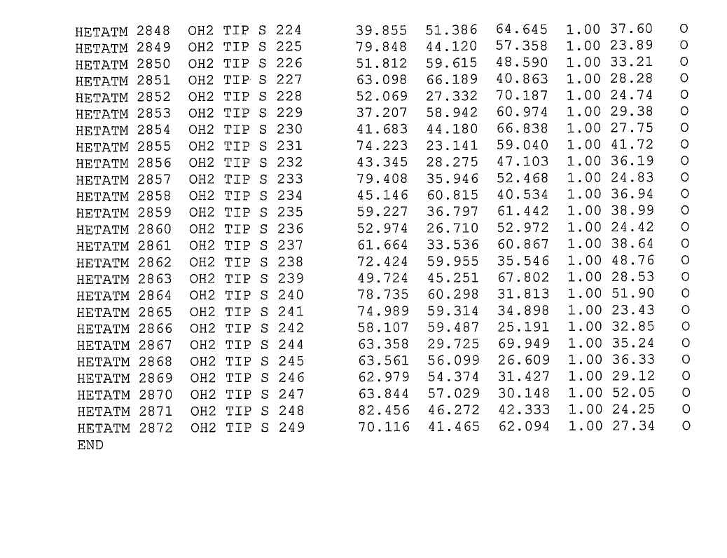

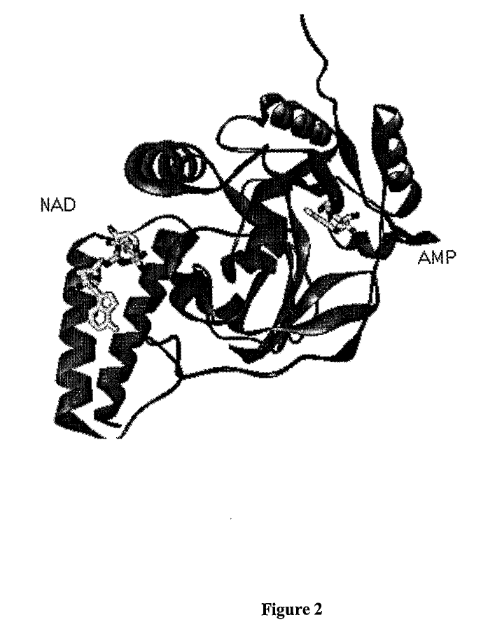

In another embodiment, the invention includes a crystallized H. influenzae LigA adenylation domain complexed with NAD⁺ and AMP, and characterized by the atomic coordinates presented in FIG. 1. In the present invention, the crystals can diffract to about 1.7 Å.

One example of the crystallized complex is characterized as belonging to the tetragonal space group P4₃2₁2 and having cell parameters of a=b=(70.23+/−0.7) Å, c=(161.28+/−0.3) Å and α=β=γ=(90.00)°.

Methods of making crystals are known in the art. In one example, a crystallized complex, as described above, can be produced by the process of preparing a first solution containing H. influenzae LigA adenylation domain of adequate purity, for example >95%, and in an appropriate buffer, for example 50 mM Tris-HCl pH8.5; preparing a second solution containing a suitable precipitant, for example a salt or polyethylene glycol; combining the first solution and the second solution, thereby producing a combination; and forming drops from the combination in a method of crystallization such that the LigA adenylation domain is brought into a state of supersaturation, whereby, crystals of the LigA adenylation domain are produced.

### LigA Crystal Structure and Mode of Action

DNA ligases catalyze the formation of a phosphodiester linkage at single-strand breaks between adjacent 3′-OH and 5′-phosphate termini in double-stranded DNA. The first step of DNA ligation in bacteria requires adenylation of the ε-NH₂ group of lysine (K) in the conserved KXDG motif by the NAD⁺ substrate. This first step creates an adenylated enzyme intermediate with AMP covalently bound to the enzyme with release of nicotinamide mononucleotide (NMN). In the second step of the reaction, the adenylate moiety is transferred from the lysine residue to the terminal 5′-phosphate at the DNA nick. A phosphodiester linkage is then formed between the 5′-phosphate and the adjacent 3′-hydroxyl, producing the sealed DNA strand.

H. influenzae LigA is composed of four distinct domains: the N-terminal adenylation Domain 1, the oligomer-binding Domain 2, the zinc finger and helix-hairpin-helix motif containing Domain 3 and the C-terminal BRCA1-like Domain 4. The structure reported herein comprises an N-terminal adenylation domain (Domain 1). The asymmetric unit of the H. influenzae LigA adenylation domain crystal consists of one monomer of the polypeptide chain. Each molecule of H. influenzae LigA adenylation domain consists of two subdomains: a helix-turn-helix subdomain 1a (residues 1-58) and an ‘adenylation’ subdomain 1b (residues 59-324) formed by two anti parallel β-sheets flanked on both sides by α-helices. The AMP is covalently bound to the subdomain 1b.

### Binding Sites

The term “binding site” refers to a specific region (or atom) of the LigA adenylation domain that enters into an interaction with a molecule that binds to the LigA adenylation domain. A binding site can be, for example, a conserved structural element or a combination of several conserved structural elements, a substrate binding site, a cofactor binding site, an activator binding site, an inhibitor binding site, an allostearic binding site, or an intermolecular interface.

A substrate binding site includes a specific region (or atom) of the LigA adenylation domain that interacts with a substrate, such as AMP. A substrate binding site may comprise, or be defined by, the three dimensional arrangement of one or more amino acid residues within a folded polypeptide. The substrate can be a naturally-occurring or artificial compound. In one embodiment of the invention, the substrate binding site for H. influenzae LigA adenylation domain includes the amino acids Ser81, Leu82, Glu114, Lys116, Gly119, Arg137, Tyr226 and Val289 of SEQ ID NO:1. In another embodiment of the invention, the substrate binding site for H. influenzae LigA adenylation domain includes the amino acids Tyr18, Glu19, Tyr22, Val30, Pro31, Asp32, His23, Tyr35, Asp36, Phe39, His40, Lys43, Thr59 and Arg154 of SEQ ID NO:1.

An inhibitor binding site includes a specific region (or atom) of LigA adenylation domain that interacts with an inhibitor that acts to prevent LigA activity. An inhibitor binding site may comprise, or be defined by, the three dimensional arrangement of one or more amino acid residues within a folded polypeptide. In the present invention, an inhibitor can be a compound that can compete or otherwise prevent LigA activity, e.g., the compound can bind to the substrate binding site on LigA.

### Machine Readable Data Storage Medium

The list of atomic coordinates defining the LigA adenylation domain crystal structure can be stored electronically, for example on a machine readable storage medium, such as a disk, so that the coordinates may be accessed and manipulated by a computer. For example, using 3D-visualisation software it is possible to depict the structure represented by the atomic coordinates on a computer graphics screen and to study hypothetical interactions with candidate inhibitors. In this way, the atomic coordinates of this invention are a useful tool for the design of novel inhibitors that are candidates for new antibacterial agents.

### Computer-Assisted Methods of Identifying LigA Binding Agents

The present invention includes a computer-assisted method for identifying a potential LigA binding agent such as a modifier, particularly a potential inhibitor of LigA activity.

Those of skill in the art will understand that a set of atomic co-ordinates, such as those tabulated in FIG. 1, may be manipulated mathematically, for example by rotation or translation, such that an entirely different set of atomic co-ordinates from those presented in FIG. 1 define a similar or identical shape and thus represent the same invention.

The crystal structure of the LigA adenylation domain, and the binding sites described herein are useful for the design of agents, particularly selective inhibitory agents, which inhibit LigA, and, thus, could act as antibacterial agents. In a related embodiment, the present invention encompasses a method for structure-based drug design of an agent that inhibits LigA activity.

More particularly, the design of compounds that inhibit LigA according to this invention generally involve consideration of two factors. First, the compound must be capable of physically and structurally associating with the LigA adenylation domain via covalent and/or non-covalent interactions. Non-covalent molecular interactions important in the association of LigA with its substrates, allosteric effectors, or inhibitor, include hydrogen bonding, van der Waals and hydrophobic interactions.

Second, the compound must be able to assume a conformation that allows it to associate with the LigA adenylation domain. Although certain portions of the compound will not directly participate in this association with LigA, those portions may still influence the overall conformation of the molecule. This, in turn, may have a significant impact on potency. Such conformational requirements include the overall three-dimensional structure and orientation of the chemical entity or compound in relation to all or a portion of a binding site, e.g., a substrate binding site, a cofactor binding site, an intermolecular interface of LigA, or the spacing between functional groups of a compound comprising several chemical entities that directly interact with LigA.

The potential inhibitory effect of a chemical compound on LigA may be estimated prior to its synthesis and testing by the use of computer modeling techniques. If the theoretical structure of the given compound suggests insufficient interaction and association between it and the LigA adenylation domain, synthesis and testing of the compound is obviated. However, if computer modeling indicates a strong interaction, the molecule may then be synthesized and tested for its ability to bind to LigA in a suitable assay. In this manner, synthesis of inactive compounds may be avoided.

One embodiment of the present invention relates to any computer-assisted method using known binding agents of LigA, such as AMP or NAD⁺ to determine the fit of a known agent for comparison to a candidate inhibitor.

In a specific embodiment, the computer-assisted method of identifying an agent that is a binding agent of LigA comprises the steps of (1) supplying the computer modeling application the atomic coordinates of a known agent that binds a binding site on LigA, such as a substrate of LigA that binds a substrate binding site of; (2) supplying the computer modeling application the atomic coordinates of the LigA adenylation domain as provided in FIG. 1, or alternatively, atomic coordinates having a root mean square deviation from the atomic coordinates of FIG. 1 with respect to conserved backbone atoms of the listed amino acid sequence of not more than 1.0 Å, or a root mean square deviation of not more than 1.5 Å; (3) quantifying the fit of an agent that binds the binding site of LigA; (4) supplying the computer modeling application with a set of atomic coordinates of an agent to be assessed to determine if it binds a binding site of LigA; (5) quantifying the fit of the test agent in the binding site using a fit function; (6) comparing the fit calculation for the known agent with that of the test agent; and (7) selecting a test agent that has a fit better than, or approximates, the fit of the known agent. For example, the atomic co-ordinates of the known binding agent used in the method above can be those of an NAD⁺ molecule bound to the substrate binding site present on the LigA adenylation domain of the invention as defined by the atomic coordinates tabulated in FIG. 1. The fit of the NAD⁺ molecule to the binding site of the LigA adenylation domain can be quantified by calculating the surface area on both the NAD⁺ molecule and the LigA adenylation domain molecule which is removed from solvent (buried surface) upon binding of the NAD⁺ to the binding site, using, for example, a program such as Areaimol (CCP4, 1994, supra). The ratio of these two values provides an estimation of the surface or shape complementarity of NAD⁺ to the binding site of LigA. The fit of a test agent which may bind to the same or similar binding site of the LigA adenylation domain as NAD⁺, can then be compared to the fit of by, for example, docking of the test agent into the binding site of the LigA adenylation domain where NAD⁺ is observed to bind, and again performing a calculation to compare the surface area on both the test agent and the LigA adenylation domain molecules that is removed from solvent upon binding of the test agent. A ratio of the buried surface areas that is closer to unity may indicate a better fit.

Another approach made possible by this invention, is to screen computationally small molecule databases for chemical entities or compounds that can bind in whole, or in part, to a binding site of the LigA adenylation domain. In this screening, the quality of fit of such entities or compounds to the binding site may be judged either by shape complementarity (DesJarlais et al., 1988, J. Med. Chem. 31:722-729) or by estimated interaction energy (Meng et al., 1992, J. Comp. Chem., 13:505-524).

Methods to screen chemical entities or fragments for their ability to associate with the LigA adenylation domain and more particularly with the individual binding sites of the LigA adenylation domain are known in the art. Such methods can include the use of computers in a process known as docking. Docking may be accomplished using software such as Quanta and Sybyl, followed by energy minimization and molecular dynamics with standard molecular mechanics forcefields using software such as CHARMM and AMBER.

Specialized computer programs may also assist in the process of selecting fragments or chemical entities. These include:
1. GRID (Goodford, 1985, J. Med. Chem., 28:849-857). GRID is available from Oxford University, Oxford, UK;
2. MCSS (1991, Miranker and Karplus, Proteins: Structure, Function and Genetics, 11:29-34). MCSS is available from Molecular Simulations, Burlington, Mass.;
3. AUTODOCK (Goodsell and Olsen, 1990, Proteins: Structure, Function and Genetics, 8:195-202). AUTODOCK is available from Scripps Research Institute, La Jolla, Calif.; and
4. DOCK (Kuntz et al., 1982, J. Mol. Biol., 161:269-288). DOCK is available from University of California, San Francisco, Calif.

Additional commercially available computer databases for small molecular compounds include the Cambridge Structural Database and the Fine Chemical Database (Rusinko, 1993, Chem. Des. Auto. News, 8:44-47).

Once suitable chemical entities or fragments have been selected, they can be assembled into a single compound or inhibitor. Assembly may be proceeded by visual inspection of the relationship of the fragments to each other on the 3D image displayed on a computer screen in relation to the structure/atomic coordinates of the LigA adenylation domain. This would be followed by manual model building using software such as Quanta or Sybyl.

Useful programs to aid one of skill in the art in connecting the individual chemical entities or fragments include:
1. CAVEAT (Bartlett et al., 1989, in Molecular Recognition in Chemical and Biological Problems, Special Pub., Royal Chem. Soc., 78:182-196). CAVEAT is available from the University of California, Berkeley, Calif.;
2. 3D Database systems such as MACCS-3D (MDL Information Systems, San Leandro, Calif.) This area is reviewed in Martin, 1992, Med. Chem., 35:2145-2154; and
3. HOOK (available from Molecular Simulations, Burlington, Mass.).

Instead of proceeding to build a LigA inhibitor in a step-wise fashion one fragment or chemical entity at a time as described above, inhibitory or other types of binding compounds may be designed as a whole or “de novo” using either an empty active site or optionally including some portion(s) of a known inhibitor(s). These methods include:
1. LUDI (Bohm, J. Comp. Aid. Molec. Design 6:61-78, 1992). LUDI is available from Biosym Technologies, San Diego, Calif.; and
2. LEGEND (Nishibata and Itai, Tetrahedron, 47:8985, 1991). LEGEND is available from Molecular Simulations, Burlington, Mass.
3. LeapFrog (available from Tripos Associates, St. Louis, Mo.)

The potential interference of the candidate inhibitor with the activity of LigA adenylation domain is assessed and the candidate inhibitor is structurally modified as needed to produce a set of atomic coordinates for a modified candidate inhibitor. The modified candidate inhibitor is further assessed, using computer-assisted techniques and, optionally, in vitro and/or in vivo testing and modified further, if needed, to produce a modified candidate inhibitor with enhanced properties (e.g., greater inhibitory activity than the starting candidate inhibitor).

A variety of conventional techniques may be used to carry out each of the above evaluations as well as the evaluations necessary in screening a candidate compound for ability to inhibit LigA. Generally, these techniques involve determining the location and binding proximity of a given moiety, the occupied space of a bound inhibitor, the amount of complementary contact surface between the inhibitor and protein, the deformation energy of binding of a given compound and some estimate of hydrogen bonding strength and/or electrostatic interaction energies. Examples of techniques useful in the above evaluations include: quantum mechanics, molecular mechanics, molecular dynamics, Monte Carlo sampling, systematic searches and distance geometry methods (Marshall, Ann. Rev. Pharmacol. Toxicol., 27:193, 1987). Specific computer software has been developed for use in carrying out these methods. Examples of programs designed for such uses include:

Gaussian 92 [M. J. Frisch, Gaussian, Inc., Pittsburgh, Pa. ©1993]; AMBER [P. A. Kollman, University of California at San Francisco, ©1993]; QUANTA/CHARMM [Molecular Simulations, Inc., San Diego, Calif., ©1992]. Other molecular modeling techniques may also be employed to screen for inhibitors of X. See, for example, Cohen et al., 1990, J. Med. Chem., 33:883-894; Navia & Murcko, 1992, Curr. Opin. Struct. Biol., 2:202-210. The model building techniques and computer evaluation systems described herein are not a limitation on the present invention, but all depend for their timely execution on the availability of the atomic coordinates of the LigA adenylation domain as provided in FIG. 1.

Other hardware systems and software packages will be known and of evident applicability to those skilled in the art.

Thus, using these computer evaluation systems, a large number of compounds may be quickly and easily examined and expensive and lengthy biochemical testing avoided. Moreover, the need for actual synthesis of many compounds is effectively eliminated.

In another embodiment, the present invention relates to a method of making a candidate modifier of the LigA by chemical, enzymatic or other synthetic methods. Candidate modifiers identified or designed as described herein can be made using techniques known to those of skill in the art.

### In Vitro and In Vivo Binding Analysis

Methods of the invention include methods for identifying inhibitors of LigA using the crystal structure and novel binding sites described herein. Inhibitors included in the invention include any inhibitor that can bind to all, or a binding site, of LigA, and may be competitive or non-competitive inhibitors. Once identified and screened for biological activity, these inhibitors may be used therapeutically or prophylactically to block bacterial growth and spread.

One design approach is to probe the LigA of the invention with molecules composed of a variety of different chemical entities to determine optimal sites for interaction between candidate LigA binding agents and LigA. For example, high resolution X-ray diffraction data collected from crystals soaked with solvent allows the determination of where each type of molecule binds. As used herein, the term “soaked” refers to a process in which the crystal is transferred to a solution containing the compound of interest, for example an organic solvent, an inhibitor, a substrate or an allosteric modulator. Small molecules that bind tightly to those sites can then be designed, synthesized and tested for their LigA inhibitory activity (Bugg et al., 1993, Scientific American, December: 92-98; West et al., 1995, TIPS, 16:67-74).

The LigA of the invention may also be used to confirm the binding, and provide information on the binding mode of agents identified by, for example, any of the computer modeling methods described herein, in vitro binding assays, or high throughput screening. For example, high resolution diffraction data collected from crystals of LigA grown in the presence of the proposed binding agent can be used in combination with the LigA atomic coordinates tabulated in FIG. 1, to obtain the structure of the complex between LigA and the proposed binding agent using the method of molecular replacement as described below. Alternatively, the atomic coordinates of the LigA adenylation domain molecules listed in FIG. 1 may be used directly in combination with the experimental X-ray diffraction data to generate a difference Fourier electron density map from which the binding of the agent can be identified. Pre-existing crystals of the LigA adenylation domain may alternatively be transferred to a solution containing the proposed binding agent for a length of time sufficient to allow the agent to diffuse through the crystal lattice and bind to a binding site of LigA. X-ray diffraction data can then be collected from these crystals and used as described above to determine the nature of the binding of the agent to LigA. These methods provide confirmation of the binding of the agent to the LigA adenylation domain, and additionally elucidate the nature of any interactions between the LigA adenylation domain and the binding agent, thus permitting further rounds of optimisation of the binding agent.

The LigA adenylation domain data of the invention may also be used in combination with, for example data from NMR spectroscopic experiments, to confirm the binding of agents identified by any of the computer modelling methods described above or by any other methods, for example in vitro binding assays, or high throughput screening. For example, measurement of changes in NMR chemical shifts for samples of LigA analysed in the presence and absence of the binding agent allows determination of the binding affinity of the agent (K_{D}) for LigA. Further, mapping of the residues giving rise to the changes in chemical shift onto the structure of the LigA of the invention allows identification of the binding site for the agent of interest.

Once identified by the techniques described herein, the inhibitor may be tested for LigA binding and inhibitory bioactivity using standard techniques. For example, LigA may be used in binding assays using conventional formats to screen inhibitors. Suitable assays for use include, but are not limited to, the enzyme-linked immunosorbant assay (ELISA) or a fluorescence quench assay. Other assay formats may be used, for example a coupled assay in which generation of product may be spectrophotometrically detected; these assay formats are not a limitation on the present invention.

The present invention also includes an in vivo analysis of the LigA activity of the test binding agents.

### Homology Modelling

In certain embodiments the present invention relates to a method for generating 3-D atomic coordinates of a protein homologue or a variant of H. influenzae LigA using the atomic coordinates of H. influenzae LigA adenylation domain described in FIG. 1, comprising,

a. identifying one or more polypeptide sequences homologous to H. influenzae LigA adenylation domain;

b. aligning the sequences with the sequence of H. influenzae LigA adenylation domain which comprises a polypeptide with the amino acid sequence of SEQ ID NO:1;

c. identifying structurally conserved and structurally variable regions between the homologous sequence(s) and H. influenzae LigA adenylation domain;

d. generating 3-D atomic coordinates for structurally conserved residues of the homologous sequence(s) from those of H. influenzae LigA adenylation domain using atomic coordinates of H. influenzae LigA adenylation domain, such as those listed in FIG. 1;

e. generating conformations for helices, strands, loops, and/or turns in the structurally variable regions of the homologous sequence(s);

f. building side-chain conformations for the homologous sequence(s); and

g. combining the 3-D atomic coordinates of the conserved residues, loops and side-chain conformations to generate full or partial 3-D atomic coordinates for the homologous sequence(s).

Thus, the LigA adenylation domain structure described herein allows the modeling of structures of homologous proteins for which experimental structural information cannot be easily obtained.

### Molecular Replacement

The LigA adenylation domain may crystallize in more than one form. Therefore, the atomic coordinates of the LigA adenylation domain as described herein are particularly useful to solve the structure of additional crystal forms of the LigA adenylation domain, or binding domains of additional crystal forms of the LigA adenylation domain. Portions of the LigA adenylation domain of the present invention function as the active site (substrate binding site). They may also be used to solve the structure of the LigA adenylation domain mutants, the LigA adenylation domain complexes, the LigA adenylation domain isozymes or of the crystalline form of other proteins with significant amino acid sequence homology or structural homology to the LigA adenylation domain. In one embodiment, significant amino acid sequence identity comprises at least 35%, 45%, 50%, 54%, 60%, 65%, 70%, 75%, 80%, 85%, 90%, 95%, 98%, or 99% identity to any functional domain of the LigA adenylation domain. For example, the amino acid sequence identity for LigA in the gram negative bacteria of E. coli, H. pylori and Pseudomonas aeruginosa is 61%, 37% and 55%, respectively, and the sequence similarity is around 76%, 59% and 68% respectively. Moreover, the amino acid sequence identity for LigA in the gram positive bacteria of Streptococcus and Staphylococcus is around 40% and the sequence similarity is around 60%. An example of structural homology would be other members who have similar functional fold classification. Such members can be readily identified using scop (see http://scop.berkeley.edu/).

One method that may be employed for this purpose is molecular replacement. In this method, the unknown crystal structure, whether it is another crystal form of H. influenzae LigA adenylation domain or the crystal of some other protein with significant amino acid sequence homology to the LigA adenylation domain, may be determined using the LigA adenylation domain atomic coordinates of this invention. This method will provide an accurate structural form for the unknown crystal more quickly and efficiently than attempting to determine such information ab initio.

Examples of programs that may be used to carry out the steps of molecular replacement include MOLREP (Vagin and Teplyakov, 1997, J. Appl. Cryst., 30:1022-1025), AMoRe (Navaza, 2001, Acta Cryst., D57 (10):1367-1372), Beast (Read, 2001, Acta Cryst., D57 (10):1373-1382), GLRF (Tong & Rossmann, 1990, Acta Cryst., A46:783-792), COMO (Jogl et al., 2001, Acta Cryst., D57 (8):1127-1134), EPMR (Kissinger et al., 1999, Acta Cryst., D55 (2):484-491). The MOLREP, AMoRe and Beast software are distributed as part of the CCP4 software package (CCP4, Acta Cryst., D50:760-763, 1994). As an example, MOLREP is an integrated molecular replacement program that finds molecular replacement solutions using a two-step procedure: (1) rotation function (RF) search to identify the orientation of the model and (2) cross translation function (TF) and packing function (PF) search to identify the position of the oriented model. The translation function checks several peaks of the rotation function by computing a correlation coefficient for each peak and sorting the result. The packing function is important in removing incorrect solutions that correspond to overlapping symmetry. MOLREP can be set to search for any number of molecules per asymmetric unit and will automatically stop when no further improvement of the solution can be achieved by adding additional molecules.

In another aspect, the present invention provides a method involving molecular replacement to obtain structural information about a molecule or molecular complex of unknown structure using the software programs described above, or equivalent programs known to those skilled in the art, and the atomic coordinates described herein and tabulated in FIG. 1.

### Practice of the Invention

The practice of the present invention employs, unless otherwise indicated, conventional techniques of cell biology, cell culture, molecular biology, microbiology and recombinant DNA manipulation, X-ray crystallography, NMR spectroscopy and molecular modeling which are within the skill of the art. Such techniques are explained fully in the literature. See, for example, Molecular Cloning: A Laboratory Manual, 2nd Ed., ed. Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Crystallography made crystal clear: a guide for users of macromolecular models (Gale Rhodes, 2nd Ed. San Diego: Academic Press, 2000).

### Equivalents

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with the true scope and spirit of the invention being indicated by the claims.

The invention is further illustrated by way of the following examples, which are intended to elaborate several embodiments of the invention. These examples are not intended to, nor are they to be construed to, limit the scope of the invention.

### EXAMPLES

### Cloning of H. influenzae LigA Adenylation Domain

The H. influenzae ligA adenylation domain DNA was cloned from the sequenced Rd strain KW20 (Fleischmann et al. 1995. Science 269:496). Cloning was accomplished using specifically designed primers [HI1100-F (5′) and HI1100-R (3′)] to PCR amplify the protein coding sequence corresponding to the H. influenzae LigA adenylation domain. An NdeI restriction endonuclease (RE) site containing a start codon (ATG) was engineered into the 5′ primer (HI1100-F), and an EcoRI RE site and a stop codon (TAG) was engineered into the 3′ primer (HI1100-R). The placement of the start codon was based on alignments with other sequenced LigA genes. This is significant since the annotated H. influenzae ligA gene (HI1100) contained 27 additional nucleotides 5′ to the start codon in the present invention. The placement of the stop codon at the C-terminus of the H. influenzae LigA adenylation domain was based on the alignment of sequences from the previously published functional adenylation domains of Bacillus stearothermophilus (Timson and Wigley. 1999. J. Mol. Biol. 285:73) and Staphylococcus aureus (Kaczmarek et al. 2001. J. Bacteriol. 183:3016). A highly conserved hydrophobic residue (Leucine #324) was selected to be the final amino acid of the H. influenzae ligA adenylation domain.

The two primers used for PCR amplification were as follows:

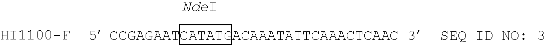

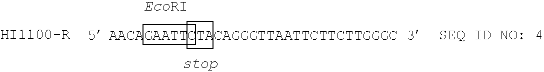

After PCR amplification of the H. influenzae adenylation domain sequence, the resulting DNA was gel purified using the Concert Rapid Gel Extraction System (Marligen Bioscience Inc.) and cloned into pGEM-T (Promega). Positive DNA clones were chosen for DNA sequencing in order to confirm the exact DNA sequence identity. The DNA insert in pSM156 encoded a 324 amino acid polypeptide that was 100% identical to amino acids 10-333 of the annotated H. influenzae LigA (HI1100).

Expression of H. influenzae LigA Adenylation Domain

The H. influenzae ligA adenylation domain insert was subcloned into the NdeI and EcoRI RE sites of expression vector pET30a (Novagen, EMD Biosciences, Inc.) to make pSM158. The DNA of the insert and junctions in pSM158 was sequenced to confirm its identity. pSM158 was transformed into E. coli BL21 (DE3) cells for over-expression. Expression was carried out at 30° C. in a 100 mL culture. Protein expression was induced by adding 1 mM Isopropylthio-β-D-galactoside (IPTG) when the cell density of the growing culture reached an OD₆₀₀ between 0.35 and 0.5. The cells were collected by centrifugation, and chilled to 4° C. after growing for 2 additional hours. The induced cells expressed a protein with an apparent molecular mass of 36,000 Daltons as determined by SDS-polyacrylamide gel electrophoresis, which is consistent with the size expected for the H. influenzae LigA adenylation domain. Protein expression in E. coli BL21(DE3) cells containing the H. influenzae ligA adenylation domain clone was scaled-up to six-1 L cultures according the above conditions. Cell paste was collected by centrifugation and stored at −20° C. until use.

Nucleotide sequence of ligA:

(SEQ ID NO:2)

ATGACAAATATTCAAACTCAACTAGACAATCTACGCAAAACCTTGCGCCA
ATATGAATACGAATACCACGTTTTAGATAATCCGAGTGTGCCTGATAGCG
AATACGATCGTTTATTTCATCAGCTCAAAGCCCTAGAATTAGAGCATCCT
GAATTTCTGACGTCAGATTCGCCCACTCAACGTGTTGGTGCAAAACCACT
TTCTGGGTTTAGCCAAATTCGTCACGAAATTCCTATGCTCTCTTTGGATA
ATGCTTTTTCCGATGCAGAATTTAATGCTTTTGTAAAACGCATTGAAGAT
CGTTTAATCCTATTACCGAAACCACTTACTTTCTGTTGCGAACCTAAACT
TGATGGCTTGGCTGTGAGTATTTTGTATGTTAATGGTGAACTTACACAAG
CCGCCACTCGTGGTGATGGCACCACAGGCGAAGATATTACAGCCAATATC
CGCACGATTCGTAATGTTCCATTGCAACTTTTAACAGATAATCCTCCAGC
ACGTTTAGAGGTGCGGGGCGAAGTTTTTATGCCGCACGCAGGCTTTGAGC
GTTTAAATAAATATGCGTTAGAACATAATGAAAAAACCTTTGCTAATCCT
CGCAATGCAGCGGCAGGCTCTTTACGCCAGCTTGATCCTAATATTACCAG
CAAACGTCCGCTGGTATTAAATGCTTATGGTATTGGAATTGCTGAGGGGG
TTGATCTGCCGACTACGCATTATGCTCGTTTGCAATGGCTAAAATCTATC
GGGATTCCAGTAAATCCTGAAATTCGTTTATGCAATGGTGCAGATGAAGT
TTTAGGTTTTTATCGAGATATTCAAAACAAACGTAGCTCGTTAGGTTATG
ATATTGACGGAACGGTATTAAAAATCAATGATATAGCCTTACAAAATGAA
CTAGGATTTATTTCTAAAGCACCTCGCTGGGCGATTGCTTATAAATTCCC
CGCCCAAGAAGAATTAACCCTGTAG
Amino acid sequence of LigA adenylation domain:

(SEQ ID NO:1)

MTNIQTQLDNLRKTLRQYEYEYHVLDNPSVPDSEYDRLFHQLKALELEHP
EFLTSDSPTQRVGAKPLSGFSQIRHEIPMLSLDNAFSDAEFNAFVKRIED
RLILLPKPLTFCCEPKLDGLAVSILYVNGELTQAATRGDGTTGEDITANI
RTIRNVPLQLLTDNPPARLEVRGEVFMPHAGFERLNKYALEHNEKTFANP
RNAAAGSLRQLDPNITSKRPLVLNAYGIGIAEGVDLPTTHYARLQWLKSI
GIPVNPEIRLCNGADEVLGFYRDIQNKRSSLGYDIDGTVLKINDIALQNE
LGFISKAPRWAIAYKFPAQEELTL

Purification and Characterization of H. influenzae DNA Ligase Adenylation Domain

The frozen cell paste was suspended in 60 ml of Lysis Buffer [25 mM Tris-HCl, pH 8.0, 2 mM EDTA, 5 mM DTT, 10% Glycerol, 1 mM PMSF, 1 Protease inhibitor cocktail tablet (Roche Molecular Biochemical)]. Cells were disrupted by passing them twice through a French press operated at 18,000 psi, and the crude extract was centrifuged at 25,000 rpm (45 Ti rotor, Beckman) for 30 min at 4° C. The supernatant was loaded at a flow rate of 1.5 ml/m onto a 20 ml Q-Sepharose HP (HR16/10) column (Pharmacia) pre-equilibrated with Buffer A (25 mM Tris-HCl, pH 8.0, 2 mM EDTA, 5 mM DTT, 10% Glycerol). The column was then washed with Buffer A, and the protein was eluted by a linear gradient from 0 to 1 M NaCl in Buffer A. Fractions containing ligase were pooled, and 3 M (NH₄)₂SO₄ in 25 mM Tris/HCl, pH 8.0, 2 mM EDTA, 5 mM DTT, 10% Glycerol was added to a final concentration of 1 M. The sample was applied at a flow rate of 1.5 ml/min to a 20 ml Phenyl Sepharose HP (HR16/10) column (Pharmacia) pre-equilibrated with Buffer B [25 mM Tris-HCl, pH 8.0, 2 mM EDTA, 5 mM DTT, 10% Glycerol, 1M (NH₄)₂SO₄]. The column was washed with Buffer B, and the protein was eluted by a linear gradient from 1 to 0 M (NH₄)₂SO₄ in Buffer A. Fractions containing ligase were pooled, and solid (NH₄)₂SO₄ (0.4 g/ml) was added to precipitate all the proteins and mixed on ice for 1 hour. The sample was centrifuged at 25,000 rpm for 30 min at 4° C. (45 Ti rotor, Beckman), the pellet was then dissolved in 10 ml of Buffer A. The 10 ml sample was applied at a flow rate of 1.5 ml/min to a 320 ml Sephacryl S-100 (HR 26/60) (Pharmacia) pre-equalibrated with Buffer C (25 mM Tris-HCl, pH 8.0, 2 mM EDTA, 5 mM DTT, 10% Glycerol, 150 mM NaCl). The fractions containing ligase were pooled and dialyzed against 1 L Storage Buffer (10 mM Tris-HCl, pH 8.0, 0.1 mM EDTA, 100 mM KCl, 2 mM DTT, 20% Glycerol). The protein was characterized by SDS-PAGE analysis and analytical LC-MS. The determined mass of the protein indicated that the ligase was adenylated and the N-terminal methionine of the partial ligase predicted from the DNA sequence was not present [expected MW=36801.5 Da, observed=36800.0 Da]. The protein was stored at −80° C.

Crystallization of H. influenzae DNA Ligase (LigA) Adenylation Domain.

Purified H. influenzae LigA adenylation domain (adenylated at Lys116) was subjected to sparse matrix crystallization screening, using a protein concentration of about 40 mg/ml in 1 mM Tris-HCl pH 7.5 at a temperature of 290 K. Screening leads were optimized using standard techniques.

Crystals having the atomic coordinates of FIG. 1 were obtained by vapor diffusion using the hanging drop method (see, for example, “Protein Crystallization”, Terese M. Bergfors (Ed.), International University Line, pp 7-15, 1999). Purified H. influenzae LigA adenylation domain had been stored at 193 K at a concentration of 36 mg/ml in Storage Buffer (10 mM Tris-HCl, pH 8.0, 0.1 mM EDTA, 100 mM KCl, 2 mM DTT, 20% Glycerol). Single aliquots containing approximately 4.0 mg of protein were thawed from storage, and were extensively washed in 1 mM Tris-HCl pH7.5. The final protein concentration was adjusted to 40 mg/ml. Crystals were also obtained using a final protein concentration of from about 25 to about 50 mg/ml, however the size of the crystals were much smaller with the higher and lower protein concentrations. The reservoir solution typically contained 16% (w/v) polyethylene glycol (PEG) 3500 and 350 mM sodium potassium tartrate. The concentration of PEG3500 in the reservoir solution could be varied from about 14% (w/v) to about 30% (w/v), and crystals obtained by corresponding adjustment of the protein concentration in the protein solution, or the ratio of protein solution to reservoir solution in the hanging drop. Similarly, the concentration of sodium potassium tartrate in the reservoir solution could be varied from about 200 mM to about 400 mM, with optimal results observed from about 300 to about 350 mM concentration.

Hanging drops were set up by mixing 2 microliters of protein solution with 2 microliters of the reservoir solution and suspending the drop over 500 microliters of reservoir solution. Crystals of dimensions up to 200×200×100 microns were observed to grow within 3-4 days at a temperature of 290 K. The size of the hanging drop, and the ratio of protein to reservoir solution may also be varied.

The crystals were equilibrated in drops containing a cryoprotectant solution before flash freezing in liquid nitrogen for transport and data collection. The selected crystals were treated with the cryoprotectant solution in the following manner. Stock solutions containing the reservoir solution corresponding to the selected drop supplemented with 5, 10, 15 and 20% (v/v) glycerol were prepared. A sample from each concentration was then taken up in a 20 micron nylon crystal-mounting loop (Hampton Research, California, USA) and tested by flash cooling in liquid nitrogen for a clear glassy freeze. A nominal drop volume of 5 microliters of each concentration of glycerol was spotted onto a silicozied glass cover slide. Using the crystal-mounting loop, crystals of interest were scooped from the respective drops and gently transferred to the cryoprotectant drop with the lowest concentration of glycerol (5%). The crystals were allowed to equilibrate for 2 minute before transferring them to the next higher concentration of glycerol. After equilibration in the highest cryprotectant concentration, the crystals were removed from the drop using the crystal-mounting loop and flash frozen in liquid nitrogen. These crystals were shipped for data collection at a synchrotron radiation source.

### X-ray Diffraction Data Collection

Crystals diffracted to about 1.7 Å resolution using ESRF synchrotron radiation source at Grenoble Crystals belonged to the tetragonal space group P4₃2₁2 and had cell parameters of a=b=(70.23+/−0.7) Å, c=(161.28+/−0.3) Å and α=β=γ=(90.00)°. This crystal form is encompassed by the atomic coordinates of FIG. 1. A complete data set was collected at ESRF to 1.7 Å resolution.

### Phase Calculation Using Molecular Replacement Method

Analysis of the crystal unit cell dimensions indicated that the crystallographic asymmetric unit likely contained one molecule of LigA adenylation domain. The phase problem for the structure was solved by molecular replacement using the programs AmoRe (Navaza, 1994). A search model was generated by threading the sequence of H. influenzae LigA adenylation domain onto the previously determined structure of the same molecule from Bacillus stearothermophilus (pdb ID#: 1B04). Only data to 4 Å were used for initial cross rotation function search followed by the translation function search on the best cross function solution. The highest correlation coefficient of 60% and the lowest R-factor of 42.1% among all solutions clearly indicated the correct translation function solution of the model. In order to further refine the orientation and the position of the molecules in the unit cell of LigA adenylation domain, the initial refinement was performed in the resolution range of 4-34.9 Å using the rigid body refinement technique of CNX (Accelrys). After 30 cycles of refinement, the R-factor dropped to 39.3% and a clear and connected density observed around the model. These results indicated that indeed the model obtained from Amore was the correct solution.

Model Building and Refinement of the H. influenzae LigA Adenylation Domain Crystal Structure

Refinement of this model was continued using the program CNX (Accelrys), applying bulk solvent and overall anisotropic B-factor corrections. Iterative rounds of simulated annealing with torsion angle dynamics (starting temperature 2500 K) followed by 50 cycles of energy minimizations and finally 20 cycles of individual isotropic B-factor refinement were performed. A round of interactive model building was then carried out using the program O, Significant peak in the difference Fourier (Fo-Fc) electron density map consistent with the covalently linked adenyl moiety was observed close to (with electron density linked to) the conserved Lys116. An additional significant density in the difference Fourier electron density map that did not belong to any of the protein side-chains was found in the N terminal domain 1a. This density was interpreted as arising from a bound nucleotide. In the later stages of refinement, the electron density in this region became sufficiently clear to assign it with confidence to the NAD⁺ molecule. Adenosine and NAD⁺ moieties were included in the model at this point, followed by a further round of interactive rebuilding, to produce a model with an R-value of 0.27 and an R-free value of 0.28 was produced. Water molecules were included using the water-pick option in CNX, followed by a couple of rounds of refinement which produced a final model with an R-value of 0.21 and an R-free value of 0.23.

The R-value describes the discrepancy between the observed data and synthetic data calculated from the model. The R-free is the same, but calculated from a test set of reflections, usually 5% of total, that are set aside at the beginning of the refinement and serve as an unbiased reference to avoid over-fitting of the data. The R-value is resolution dependent but should typically be equal to or less than 0.25, and the Rfree typically not more than 5% higher.

The final model consists of one polypeptide chain of 324 amino acids, one molecule of adenosine (covalently linked to Lys116), one molecule of NAD⁺ and 263 ordered water molecules. Statistics of the final model are given in Table 4.

TABLE 2
Refinement statistics for H. influenzae
LigA adenylation domain final model

| Space group: | P4₃2₁2 |
|---|---|
| Unit Cell | a = 70.23 |
| | b = 70.23 |
| | c = 161.28 |
| Resolution limit (Å) | 1.70 |
| Resolution range (Å) | 34.9-1.70 |
| Completeness overall (%) | 86.2 |
| Multiplicity (%) | 2.8 (1.5) |
| Rmergeₒᵥₑᵣₐₗₗ¹ | 8.3 (20.6) |
| Rvalueₒᵥₑᵣₐₗₗ (%)² | 21.5 |
| Rvalue_{free} (%) | 23.8 |
| R.m.s. deviations from ideal values | |
| Bond lengths (A) | 0.06 |
| Bond angles (°) | 1.9 |
| Average B values (Å) | |
| Protein main chain atoms | 14 |
| Protein all atoms | 15 |
| Ligand | |
| NAD | 25 |
| AMP | 12 |
| Solvent | 23 |
| φ, Ψ angle distribution for residues³ | |
| In most favored regions (%) | 92.9 |
| In additional allowed regions (%) | 6.7 |
| In generously regions (%) | 0.4 |
| In disallowed regions (%) | 0 |

¹R_{merge} = Sₕₖₗ[(Σᵢ|lᵢ − <l>|)/Σᵢ lᵢ]
²Rᵥₐₗᵤₑ = Sₕₖₗ||F_{obs}| − |F_{calc}||/Sₕₖₗ|F_{obs}|
R_{free} is the cross-validation R factor computed for the test set of 5% of unique reflections
³Ramachandran statistics as defined by PROCHECK

FIG. 1 is a listing of the three-dimensional atomic coordinates of the crystal structure of LigA adenylation domain from H. influenzae complexed with adenosine and NAD⁺. In the figure, the atom listing is preceded by the heading CRYST1, which is followed by the 3 dimensions of the crystallographic unit cell. The next three values define a matrix that converts atomic co-ordinates from orthogonal Ångström coordinates to fractional coordinates of the unit cell. Each row labeled ATOM gives the (arbitrary) atom number, the label given to each amino acid main chain, each atom type, the amino acid residue type, the protein chain label and the amino acid residue number. The first three numbers in the row give the orthogonal X, Y, Z coordinates of the atom. The next number is an occupancy number and is less than 1.0 if the atom was seen in more than one position (the amino acid could be seen in more than one orientation). The final number is a temperature factor that relates to the thermal amplitude of vibrations of the atom. At the end of the listing, there are lines of data indicating the bound ligands (NAD⁺ and AMP) and ordered water molecules (HOH) included in the model.

Defining the Binding Sites of H. influenzae LigA Adenylation Domain.

Adenylated Active Site

The covalently bound AMP binding pocket is located between two β-sheets of the adenylation domain. The binding site is stabilized by a number of residues in the active site, primarily composed of five conserved motifs (I, III, IIIa, IV and V) that are characteristics of this class of nucleotidyl transferase superfamily (which include DNA ligase, RNA ligase and eukaryotic mRNA capping enzymes). Among the residues lining the binding site, the most important catalytic residue is Lys116. The AMP portion of the NAD⁺ gets covalently attached to this Lys in the first step of the reaction. A clear connected density between the α-phosphate group of the AMP to the side chain ε-N of Lys116 indicates that the AMP is indeed covalently attached to the protein in the crystal structure.

The covalently linked α-phosphate group of the bound AMP is further stabilized by electrostatic interactions with Arg201 side chain. The side chain guanidium group of Arg137 stacks over the ribose ring, with one of its nitrogen within hydrogen-bonding distance to the ring oxygen. The hydroxyl group of ribose interacts with the main chain carbonyls of Ser81 and Leu82. The adenine ring is stacked against the side chain of Tyr226 on one side, and by the side chains of Val289, Lue82 and Lys116 on the other side. Other residues that line the adenine-binding pocket include Lue117, Lys291, Glu114, and Met79. The amino group of the adenine ring is stabilized by the side chain carboxyl group of Glu114 and main chain carbonyl of Pro115. Lys116 and Glu114 form an ion pair at the base of the AMP-binding pocket. In addition to the above interactions, the C-terminal Leu324 from adjacent molecule also forms a small portion of the adenine pocket in this crystal structure. The adenosine nucleoside of the covalently linked AMP is in the anti conformation. This is in contrast to the syn conformation in other members of this superfamily, namely that of adenosine in the crystal structure of the ATP dependent T7 LigA and the guanosine in the crystal structure of eukaryotic mRNA-capping enzyme.

Residues located within a 5 Å radius of the bound AMP molecule include Ser81, Lue82, Glu114, Lys116, Gly119, Arg137, Tyr226 and Val289 of SEQ ID NO:1. All these residues are well conserved across most bacterial DNA-LigA adenylation domain. The adenylated AMP binding site of H. influenzae LigA adenylation domain thus minimally comprises residues Ser81, Lue82, Glu114, Lys116, Gly119, Arg137, Tyr226 and Val289 of SEQ ID NO:1 or in a yet further expanded definition, derived using a probe radius of 8 Å, comprises residues Met79, Leu80, Ser81, Leu82, Asp83, Asn84, Glu114, Pro115, Lys116, Leu117, Asp118, Gly119, Leu120, Ala121, Arg137, Gly138, Gly140, Arg172, Gly173, Glu174, Arg201, Ala225, Tyr226, Gly227, Asp286, Thr288, Val289, Lys291, Ala311 and Ala313.

NAD⁺ Binding Site

Based on the structural information, the NAD⁺ binding site is located between the subdomains 1a and 1b. The nicotinamide ring portion is buried into a deep pocket, whereas the remaining part of the molecule is more solvent exposed, with the residues from subdomain 1a providing majority of the interactions. The electron density of the NAD⁺ is consistent with a syn conformation for the glycosidic bond. The adenine ring binds in a pocket on the enzyme surface formed by the side-chains of Lys43, His40, Thr59, Phe39, Val62, and Arg61, and by the main-chain interactions from Thr59, Gln60, Arg61 and Val62. Direct hydrogen bonds are formed between the adenine nitrogen atoms at position N3 with the side chain nitrogen atom of His40 and at position N1 with the side chain of Thr59. The aromatic ring of Phe39 from one of the subdomain 1a helices stacks against the adenine ring and stabilizes the observed conformation. The 3′ OH group of adenosine-ribose is within hydrogen bonding distance to the side chain N of His40. The presence of His40 near the vicinity of the 2′ OH of this ribose explain the specificity of this enzyme for binding NAD (H) over NAD(H) on the basis of its ability to mediate stearic repulsion of the extra phosphate bound to the adenosyl ribose in NAD(H).

The pyrophosphate moiety of the NADH interacts with the positively charged Arg154 from subdomain 1b and hydrogen-bonding contacts mediated by water molecules, to the side-chain of His23 and Tyr35. The hydroxyl groups of the nicotinamide-ribose interact with the side-chain carboxyl groups of Asp36 and Asp32, while the ring oxygen is within hydrogen bonding distance to the Tyr22 hydroxyl group. The nicotinamide ring sits in a deep pocket, stacked between the side chain rings of Tyr22 and Tyr53. In addition, the nicotinamide pocket is bounded by the side-chains of Tyr118, Glu19, His23, Pro28, Val30 and the main chains of Ser29, Asp32, and Glu19. The amide group of nicotinamide is within hydrogen bonding distance to the main chain carbonyl group of Val30 and side chain carboxyl group of Asp32. Mutational analysis demonstrates that alanine substitutions at residues His23, Tyr35, Tyr22, Asp32 and Asp36 either significantly or completely abolish adenyl transfer from NAD⁺ without affecting the ligation of pre-formed adenylated DNA. (JBC, 277, 9695-9700). These results further provide supporting evidence that the binding site observed in this crystal structure is indeed the physiological binding pocket for NAD⁺ on LigA.

Residues located within a 5 Å radius of the bound NAD⁺ molecule include Tyr18, Glu19, Tyr22, Val30, Pro31, Asp32, Tyr35, Asp36, Phe39, His40, Lys43, Thr59 and Arg154 of SEQ ID NO:1. Most of these residues are completely conserved among 10 bacterial LigA. Thus the NAD⁺ binding site of H. influenzae LigA adenylation domain minimally comprises residues or in a yet further expanded definition, derived using an 8 Å probe radius, includes Leu15, Tyr18, Glu19, Glu21, Tyr22, His23, Pro28, Ser29, Val30, Pro31, Asp32, Ser33, Glu34, Tyr35, Asp36, Phe39, His40, Leu42, Lys43, Pro58, Thr59, Gln60, Arg61, Val62, Arg154, Ser217 and Lys218 of SEQ ID NO:1.

1. A crystal of LigA from a gram negative bacterium.

2. The crystal of claim 1, wherein the crystal is complexed with a substrate.

3. The crystal of claim 2, wherein the substrate is AMP.

4. (canceled)

5. The crystal of claim 2, wherein the substrate is NAD⁺.

6-7. (canceled)

8. The crystal of claim 1, wherein the LigA is from Haemophilus influenzae.

9. The crystal of claim 2, wherein the substrate is bound at a binding site comprising amino acid residues of Ser81, Leu82, Glu114, Lys116, Gly119, Arg137, Tyr226 and Val289 of SEQ ID NO:1.

10. The crystal of claim 2, wherein the substrate is bound at a binding site comprising amino acid residues Tyr18, Glu19, Tyr22, Val30, Pro31, Asp32, His23, Tyr35, Asp36, Phe39, His40, Lys43, Thr59 and Arg154 of SEQ ID NO:1.

11. A method of identifying a molecule that binds to LigA comprising:
a) applying a 3-dimensional molecular modeling algorithm to atomic coordinates of LigA; and
b) electronically screening stored atomic coordinates of a set of candidate compounds against the atomic coordinates of LigA to identify compounds that bind to LigA.

12. The method of claim 11, wherein the atomic coordinates are of a molecular interface of LigA.

13. The method of claim 11, wherein the atomic coordinates are given in FIG. 1.

14. A computer-assisted method of identifying an agent that is a substrate modulator of LigA, comprising:
(a) providing a computer modeling application with a set of atomic coordinates of a crystal of a LigA substrate binding site;
(b) supplying the computer modeling application with a set of atomic coordinates of an agent to be assessed to determine if it binds a substrate binding site of LigA;
(c) comparing the two sets of atomic coordinates; and
(d) determining whether the agent is expected to bind to LigA, wherein if the agent is expected to bind the substrate binding site of LigA, the agent is a substrate modulator of LigA.

15. The computer-assisted method of claim 14, wherein the set of atomic coordinates of the LigA substrate binding site is given in FIG. 1.

16. A computer-assisted method of identifying an agent that is a substrate modulator of LigA, comprising:
(a) providing a computer modeling application with a set of atomic coordinates of a crystal of LigA substrate binding site;
(b) supplying the computer modeling application with a set of atomic coordinates of an agent to be assessed to determine if it binds to the substrate binding site of LigA;
(c) comparing the two sets of atomic coordinates; and
(d) determining whether the agent is expected to bind to LigA, wherein if the agent is expected to bind the substrate binding site of LigA, the agent is a substrate modulator of LigA.

17. The computer-assisted method of claim 16, wherein the set of atomic coordinates of the substrate binding site is given in FIG. 1.
